(19) United States
(12) Patent Application Publication
(10) Pub. No.: US 2023/0149712 A1
(43) Pub. Date: May 18, 2023

(54) COMPUTATIONAL METHOD FOR CONSIDERING CONTRIBUTION OF BIOLOGICAL ACTIVITY TO COCHLEAR SENSORY AMPLIFICATION MECHANISM

(71) Applicant: Junyi LIANG, Shanghai (CN)

(72) Inventors: Junyi LIANG, Shanghai (CN); Wenjuan YAO, Shanghai (CN)

(21) Appl. No.: 17/930,881

(22) Filed: Sep. 9, 2022

(30) Foreign Application Priority Data
Nov. 12, 2021 (CN) 202111341534 .7

(51) Int. Cl. A61N 1/36; H04R 25/00

(52) U.S. Cl. CPC A61N 1/36038; H04R 25/554

(57) ABSTRACT

The present disclosure relates to the field of biophysical technology, and in particular to a computational method for considering contribution of biological activity to a cochlear sensory amplification mechanism. A new computational analysis model for motion of a key supporting structure of a cochlear sensory function considering biological activity is established based on the principle of physical mechanics. The present disclosure derives an equation of coupled motion of the basement membrane (BM) with the lymph fluid while the stiffness of the BM periodically varies in space and time, and solves it. That is, the computational method for considering contribution of biological activity to a cochlear sensory amplification mechanism is established, and the analytical method is verified by computer numerical simulation. The method established in the present disclosure is easily feasible and efficient and accurate.

### CROSS REFERENCE TO RELATED APPLICATION

This patent application claims the benefit and priority of Chinese Patent Application No. 202111341534.7, filed on Nov. 12, 2021, entitled “COMPUTATIONAL METHOD FOR CONSIDERING CONTRIBUTION OF BIOLOGICAL ACTIVITY TO COCHLEAR SENSORY AMPLIFICATION MECHANISM”, the disclosure of which is incorporated by reference herein in its entirety as part of the present application.

### TECHNICAL FIELD

The present disclosure relates to the field of biophysical technology, and in particular to a computational method for considering contribution of biological activity to a cochlear sensory amplification mechanism.

### BACKGROUND ART

Hearing loss is one of the most common human sensory disorders. 360 million people worldwide (5% of the world's population) suffer from hearing loss. Sensorineural hearing loss is the most challenging medical problem, and a cochlear active sensorineural amplification mechanism is a major problem in otology. The basement membrane (BM) is a key macroscopic structure in the cochlear sensory function. The Nobel laureate von Bexy provides a traveling wave vibration model of the BM. Previously reported cochlear computational analysis models are based on the traveling wave theory. So far, the understanding of the cochlear active amplification mechanism is limited to the interaction and energy conversion of various tissue structures in the cochlea, which amplify the motion of the BM and microscopic tissues, but the time-varying effect of itself as a material of a biologically active structure is not considered. That is, the biological activity of the BM is not considered.

### SUMMARY

An objective of the present disclosure is to solve the deficiencies in the background art by providing a computational method for considering contribution of biological activity to a cochlear sensory amplification mechanism and a battery pack architecture.

Based on the theory of physical mechanics, a dimensionless control equation for coupled motion of the endocochlear lymph fluid and the BM is established, and the motion behavior of the BM is introduced into the Navier-Stokes equation through a volume force term to couple with motion of the fluid.

To describe the biological activity of the BM by comprehensively considering a periodic function of stiffness of the BM varying with space and time, and to study the coupled vibration behavior of the BM with the lymph fluid through the periodic variation of its internal materials without external excitation.

The present disclosure adopts the following technical solutions:

A method for determining contribution of biological activity to hearing loss in a hearing-impaired person includes:

providing an analytical model, stored in a non-transitory storage device, the analytical model correlating periodic variation of a stiffness of a basement membrane in space and time, and characterizing a relationship between physical parameters of a human cochlear and an amplitude of an associate cochlear basement membrane;

using a vertical displacement of a basement membrane of a non-hearing-impaired person as an input of the analytical model to obtain a stiffness of the basement membrane of the non-hearing-impaired person;

using a vertical displacement of a basement membrane of a hearing-impaired person as an input of the analytical model to obtain a stiffness of the basement membrane of the hearing-impaired person; and

determining a hardening degree of the basement membrane of the hearing-impaired person according to the stiffness of the basement membrane of the non-hearing-impaired person and the stiffness of the basement membrane of the hearing-impaired person.

As a preferred technical solution of the present disclosure, a process of establishing the analytical model may include calculating a volume force f:

f(x,t)=∫₀^{L}K(s,t)(X₀−x)δ(x−X)ds,

in the formula, δ(x) represents a two-dimensional Dirac Delta function, X(s,t) may represents position parameters of the basement membrane (BM) in a Lagrangian coordinate, and X₀(s)=(s,0) may represent an equilibrium position;

a stiffness parameter of the BM may be assumed to vary with time and space, and may be expressed by a function as:

K(s,t)=σe^{-λs}(1+2τ sin(ωt)),

in the formula, σ may be an elastic stiffness constant in a mean time interval, λ may describe a variation of stiffness along a BM space, and from previous experimental data, the stiffness of the BM may be found to present an exponential variation rule along a length; an exponential function may be used to describe the spatial variation of the stiffness; a periodic variation rule of the stiffness in a periodic vibration process of the BM may be described by an amplitude parameter τ and a frequency ω;

according to coupled vibration interface conditions between fluid and the BM, the following may be derived:

$$\frac{\partial X}{\partial t} = u ( X , t ) = {\int }_{Ω} u ( x , t ) δ ( x - X ) dx ,$$

dimensionless processing may be used, and a dimensionless quantity may be as follows:

$$x = \frac{L \tilde{x}}{π} , t = \frac{\tilde{t}}{ω} , u = {U}_{c} \tilde{u} , p = {P}_{c} \tilde{p} , X = \frac{L \tilde{X}}{π} , s = \frac{L \tilde{s}}{π} ,$$

in the formula, a subject on a wavy line may be the dimensionless quantity, Uc and Pc may respectively represent characteristic scales of velocity and pressure, and by substituting the dimensionless quantity, the following may be obtained:

$$\begin{matrix}\frac{\partial \tilde{u}}{\partial \tilde{t}} + \tilde{u} \cdot \tilde{∇} \tilde{u} = - \tilde{∇} \tilde{p} + v \tilde{Δ} \tilde{u} + \tilde{f} \\ \tilde{∇} \cdot \tilde{u} = 0 \\ \tilde{f} ( \tilde{x} , \tilde{t} ) = {{\int }_{0}}^{π} \tilde{K} ( {\tilde{X}}_{0} - \tilde{X} ) \tilde{δ} ( \tilde{x} - \tilde{X} ) d \tilde{s} , \\ \tilde{K} ( \tilde{s} , \tilde{t} ) = κ {e}^{- α \tilde{s}} ( 1 + 2 τ sin \tilde{t} ) \\ \frac{\partial \tilde{X}}{\partial \tilde{t}} = \tilde{u} ( \tilde{X} , \tilde{t} )\end{matrix}$$

the characteristic scales of velocity and pressure may be expressed as:

$${U}_{c} = \frac{L ω}{π} , {P}_{c} = \frac{ρ {ω}^{2} {L}^{2}}{{π}^{2}} ,$$

parameters in equations may be expressed as:

$$v = \frac{μ {π}^{2}}{ρ {L}^{2} ω} , κ = \frac{σ π}{ρ L {ω}^{2}} , α = \frac{λ L}{π} ,$$

a system equation of the analytical model may be:

$$\begin{matrix}\frac{\partial u}{\partial t} = - ∇ p + v Δ u \\ ∇ \cdot u = 0\end{matrix} ,$$

and there may be the following conditions:

$$\begin{matrix}p = - κ {e}^{- α x} ( 1 + 2 τsin t ) h ( x , t ) \\ u ( x , 0 , t ) = 0 \\ v ( x , 0 , t ) = \frac{\partial h}{\partial t}\end{matrix} ,$$

h(x,t) may represent vertical displacement of the BM, p(x,t)=p(x,0⁺,t)−p(x,0⁻¹,t) may represent a pressure difference on the BM, and u(x,y,t) and v(x,y,t) may represent vertical and vertical velocities respectively.

As a preferred technical solution of the present disclosure, the solution of the analytical model may satisfy the following forms:

u(x,t)=e^{γt}P(x,t),

in the formula, the function P(x,t) represents a periodic function with a period of 2π, an exponential factor may determine the stability of the solution when t−>∞, and series representation of P(x,t) may be performed in an interval of [−π,π] to obtain:

$$\begin{matrix}u ( x , y , t ) = {e}^{γ t} {\sum }_{n = - ∞}^{∞} {\sum }_{k = - ∞}^{∞} {u}_{k}^{n} ( y ) {e}^{i n t} {e}^{ikx} \\ v ( x , y , t ) = {e}^{γ t} {\sum }_{n = - ∞}^{∞} {\sum }_{k = - ∞}^{∞} {v}_{k}^{n} ( y ) {e}^{i n t} {e}^{ikx} \\ p ( x , y , t ) = {e}^{γ t} {\sum }_{n = - ∞}^{∞} {\sum }_{k = - ∞}^{∞} {p}_{k}^{n} ( y ) {e}^{i n t} {e}^{ikx} \\ h ( x , t ) = {e}^{γ t} {\sum }_{n = - ∞}^{∞} {\sum }_{k = - ∞}^{∞} {h}_{k}^{n} ( y ) {e}^{i n t} {e}^{ikx}\end{matrix} ,$$

in the formula, Fourier series expansion in space and time may be performed on P(x,t);

parameters to be solved in the above equation may be Fourier coefficients uₖⁿ, vₖⁿ, and pₖⁿ varying along a Y-axis, and substitution may be performed to obtain an equation of pressure expressed as:

$$Δ p = {\sum }_{n , k = - ∞}^{∞} ( - {k}^{2} {p}_{k}^{n} ( y ) + {p}_{k}^{{n}^{″}} ( y ) ) {ε}_{k}^{n} = 0 ,$$

in the formula, εₖⁿ(x,t)=e^{[(γ+in)t+ikx]}, due to linear independence of εₖⁿ(x,t), there may be:

−k²pₖⁿ(y)+pₖ^{n″}(y)=0,

by solving the above equation, the following may be obtained:

$${p}_{k}^{n} ( y ) = \{ \begin{matrix}{α}_{k}^{n} {e}^{ky} , & y < 0 \\ {b}_{k}^{n} {e}^{- k y} , & y > 0\end{matrix} , and$$

$${\sum }_{n , k = - ∞}^{∞} ( γ + in ) {v}_{k}^{n} ( y ) {ε}_{k}^{n} = {\sum }_{n , k = - ∞}^{∞} ( - {p}_{k}^{{n}^{′}} ( y ) - {vk}^{2} {v}_{k}^{n} ( y ) + {vv}_{k}^{{n}^{″}} ( y ) ) {ε}_{k}^{n} ,$$

after further derivation, an ordinary differential equation may be obtained as follows:

$${v}_{k}^{{n}^{″}} ( y ) - {( {β}_{k}^{n} )}^{2} {v}_{k}^{n} ( y ) = \frac{1}{v} {p}_{k}^{{n}^{′}} ( y ) ,$$

in the formula,

$${β}_{k}^{n} = \sqrt{\frac{γ + in}{v} + {k}^{2}} ,$$

supposing γ+in≠0 and k≠0, then a solution of the above formula may be:

$${v}_{k}^{n} ( y ) = \frac{1}{2 v {β}_{k}^{n}} \{ \begin{matrix}- \frac{2 k {β}_{k}^{n}}{{( {β}_{k}^{n} )}^{2} - {k}^{2}} {α}_{k}^{n} {e}^{ky} + ( \frac{k}{{β}_{k}^{n} - k} {α}_{k}^{y} + \frac{k}{{β}_{k}^{n} + k} {b}_{k}^{y} ) {e}^{{β}_{k}^{n} y} , & y < 0 \\ \frac{2 {β}_{k}^{n}}{{( {β}_{k}^{n} )}^{2} - {k}^{2}} {b}_{k}^{n} {e}^{- ky} + ( \frac{k}{{β}_{k}^{n} - k} {b}_{k}^{y} + \frac{k}{{β}_{k}^{n} + k} {α}_{k}^{y} ) {e}^{- {β}_{k}^{n} y} , & y > 0\end{matrix} ,$$

according to continuity, an equation may be obtained:

ikuₖⁿ(y)+vₖ^{n′}(y)=0,

then the equation may be solved to obtain:

$${u}_{k}^{n} ( y ) = \frac{- i}{2 kv} \{ \begin{matrix}- \frac{2 {k}^{2}}{{( {β}_{k}^{n} )}^{2} - {k}^{2}} {α}_{k}^{n} {e}^{ky} + ( \frac{k}{{β}_{k}^{n} - k} {α}_{k}^{y} + \frac{k}{{β}_{k}^{n} + k} {b}_{k}^{y} ) {e}^{{β}_{k}^{n} y} , & y < 0 \\ - \frac{2 k}{{( {β}_{k}^{n} )}^{2} - {k}^{2}} {b}_{k}^{n} {e}^{- ky} + ( \frac{k}{{β}_{k}^{n} - k} {b}_{k}^{y} + \frac{k}{{β}_{k}^{n} + k} {α}_{k}^{y} ) {e}^{- {β}_{k}^{n} y} , & y > 0\end{matrix} ,$$

according to the continuity, interface boundary conditions may be derived:

$${u}_{k}^{n} ( {0}^{+} ) = {u}_{k}^{n} ( {0}^{-} ) = \frac{i}{2 v} ( \frac{1}{{β}_{k}^{n} + k} {α}_{k}^{n} + \frac{1}{{β}_{k}^{n} + k} {b}_{k}^{n} ) = 0 ,$$

$${v}_{k}^{n} ( {0}^{+} ) = {v}_{k}^{n} ( {0}^{-} ) = \frac{1}{2 v {β}_{k}^{n}} ( \frac{- k}{{β}_{k}^{n} + k} {α}_{k}^{n} + \frac{k}{{β}_{k}^{n} + k} {b}_{k}^{n} ) = ( γ - in ) {h}_{k}^{y}$$

after further derivation, the following may be obtained:

$${α}_{k}^{n} = - v ( γ + in ) \frac{{β}_{k}^{n} + k}{k} {β}_{k}^{n} {h}_{k}^{n} ,$$

$${b}_{k}^{n} = v ( γ + in ) \frac{{β}_{k}^{n} + k}{k} {β}_{k}^{n} {h}_{k}^{n}$$

substitution may be performed to obtain:

$${\sum }_{n , k = - ∞}^{∞} 2 v ( γ + in ) \frac{{β}_{k}^{n} + k}{k} {β}_{k}^{n} {h}_{k}^{n} {ε}_{k}^{n} = {\sum }_{n , k = - ∞}^{∞} - κ {e}^{- α x} ( 1 + 2 τ sin t ) {h}_{k}^{n} {ε}_{k}^{n} ,$$

when γ+in=0 and k=0, the above equation may be simplified as:

$$0 = {\sum }_{n , k = - ∞}^{∞} - κ {e}^{- α x} ( 1 + 2 τ sin t ) {h}_{k}^{n} {ε}_{k}^{n} ,$$

after Fourier expansion of the exponential function and sine function in the above formula, there may be 1+2 τ sin t=1−iτe^{it}+iτe^{-it};

even function periodic expansion of the exponential function may be performed, and when γ+in≠0 and k≠0, there may be:

$${\sum }_{n , k = - ∞}^{∞} 2 v ( γ + in ) \frac{{β}_{k}^{n} + k}{k} {β}_{k}^{n} {h}_{k}^{n} {ε}_{k}^{n} = {\sum }_{n , k = - ∞}^{∞} - κ ( {\sum }_{j = - ∞}^{∞} {c}_{j} {e}^{ijx} ) ( 1 - i τ {e}^{it} + i τ {e}^{- i t} ) {h}_{k}^{n} {ε}_{k}^{n} ,$$

when γ+in=0 and k=0, there may be:

$$0 = {\sum }_{n , k = - ∞}^{∞} - κ ( {\sum }_{j = - ∞}^{∞} {c}_{j} {e}^{ijx} ) ( 1 - i τ {e}^{it} + i τ {e}^{- i t} ) {h}_{k}^{n} {ε}_{k}^{n} ,$$

where

$${c}_{j} = α \frac{1 - {( - 1 )}^{j} {e}^{- α x}}{π ( {α}^{2} + {j}^{2} )}$$

may be a Fourier coefficient of the exponential function; and by sorting out coefficients of a term εₖⁿ, the following may be obtained:

$$\frac{2 {v}^{2}}{κ} ( {β}_{k}^{n} - k ) {( {β}_{k}^{n} + k )}^{2} \frac{{β}_{k}^{n}}{k} {h}_{k}^{n} + {\sum }_{j = - ∞}^{∞} {c}_{k - j} {h}_{j}^{n} = i τ {\sum }_{j = - ∞}^{∞} {c}_{k - j} ( {h}_{j}^{n - 1} - {h}_{j}^{n + 1} ) ,$$

when γ+in=0 and k=0, there may be:

$${\sum }_{j = - ∞}^{∞} {c}_{k - j} {h}_{j}^{n} = i τ {\sum }_{j = - ∞}^{∞} {c}_{k - j} ( {h}_{j}^{n - 1} - {h}_{j}^{n + 1} ) ,$$

in order to ensure space symmetry of even functions of solutions, there may be:

$${h}_{- k}^{n} = {h}_{k}^{n} \\ {h}_{k}^{- n} = \{ \begin{matrix}{\overline{h}}_{k}^{n} , & γ = 0 \\ {\overline{h}}_{k}^{n - 1} , & γ = \frac{1}{2} i\end{matrix} ,$$

for the solution u(x,t)=e^{γt}P(x,t), the following periodic conditions may be implied:

u(x,t+2πn)=e^{γ(t+2πn)}P(x,t)=ξⁿu(x,t)

if γ=0, then ξ=1, and there may be:

u(x,t+2π)=u(x,t),

the above formula may be a harmonic solution with a period of 2π; if γ=½*i, then ξ=−1, and there may be:

u(x,t+2π)=−u(x,t),u(x,t+4λ)=u(x,t),

the system equation of the analytical model may be reduced, n=0, 1, . . . , N, and k=1, 2, . . . , M, and by matrix representation, the following may be obtained:

A\vec{h}=τB\vec{h},

where

$$\vec{h} = {[ … , Re ( {h}_{k}^{n} ) , Im ( {h}_{k}^{n} ) , Re ( {h}_{k + 1}^{n} ) , Im ( {h}_{k + 1}^{n} ) , … ]}^{T} ,$$

the above equations may include 2*M*(N+1) unknown coefficients to be solved, and A and B may be skew diagonal matrices; and the skew diagonal matrix A may be expressed as A=diag(A⁰, A¹, . . . , A^{N}) and may have the following forms:

$${A}^{n} = [ \begin{matrix}{C}_{1 , 1} + {D}_{1}^{n} & {C}_{1 , 2} & … & {C}_{1 , M} \\ {C}_{2 , 1} & {C}_{2 , 2} + {D}_{2}^{n} & … & {C}_{2 , M} \\ ⋮ & ⋮ & ⋱ & ⋮ \\ {C}_{M , 1} & {C}_{M , 2} & … & {C}_{M , M} + {D}_{M}^{n}\end{matrix} ] ,$$

where

$${C}_{k , j} = [ \begin{matrix}{C}_{k - j} + {c}_{k + j} & 0 \\ 0 & {c}_{k - j} + {c}_{k + j}\end{matrix} ] , and \\ \\ \\ {D}_{k}^{n} = \frac{2 {v}^{2}}{κ k} [ \begin{matrix}Re \{ ( {β}_{k}^{n} - k ) ( {β}_{k}^{n} {+ k )}^{2} {β}_{k}^{n} \} & - Im \{ ( {β}_{k}^{n} - k ) ( {β}_{k}^{n} {+ k )}^{2} {β}_{k}^{n} \} \\ Im \{ ( {β}_{k}^{n} - k ) ( {β}_{k}^{n} {+ k )}^{2} {β}_{k}^{n} \} & Re \{ ( {β}_{k}^{n} - k ) ( {β}_{k}^{n} {+ k )}^{2} {β}_{k}^{n} \}\end{matrix} ] ,$$

the triangular skew diagonal matrix B may have the following forms:

$$B = [ \begin{matrix}\hat{B} & \hat{B} & & & \\ \hat{B} & 0 & - \hat{B} & & \\ & ⋱ & ⋱ & ⋱ & \\ & & \hat{B} & 0 & - \hat{B} \\ & & & \hat{B} & 0\end{matrix} ] , \\ where \hat{B} = [ \begin{matrix}{\hat{C}}_{1 , 1} & {\hat{C}}_{1 , 2} & … & {\hat{C}}_{1 , M} \\ {\hat{C}}_{2 , 1} & {\hat{C}}_{2 , 2} & & {\hat{C}}_{2 , M} \\ ⋮ & & ⋱ & ⋮ \\ {\hat{C}}_{M , 1} & {\hat{C}}_{M , 2} & … & {\hat{C}}_{M , M}\end{matrix} ] , \\ {\hat{C}}_{k , j} = [ \begin{matrix}0 & - {c}_{k - j} - {c}_{k + j} \\ {c}_{k - j} + {c}_{k + j} & 0\end{matrix} ] ,$$

and

both A and B matrices may be known, that is,

$${A}^{- 1} B \vec{h} = \frac{1}{τ} \vec{h} ,$$

an eigenvalue of the stability solution in the above equation may be 1/τ.

As a preferable technical solution of the present disclosure, non-periodic solution may include:

when τ=0 in the stiffness function of the BM, the stiffness function may be a non-periodic function, the solution of the equation may be stable, and the Fourier coefficient of the solution may satisfy:

$$\frac{2 ϕ γ}{k v} \sqrt{\frac{γ}{v} + {k}^{2}} ( k + \sqrt{\frac{γ}{v} + {k}^{2}} ) {h}_{k}^{0} + {\sum }_{j = - ∞}^{∞} {c}_{k - j} {h}_{j}^{0} = 0 ,$$

when k=0, there may be:

$${\sum }_{j = - ∞}^{∞} {c}_{k - j} {h}_{j}^{0} = 0 ,$$

in the above formula, ϕ=v²/κ=π³μ²/(ρσL³) may represent a ratio of a fluid viscosity resistance to an elastic force of the BM; and the above equation may be expressed as

$$T {h}^{\to 0} = 0 ,$$

and the T matrix may depend on parameters ϕ, γ, and α, and a condition for existence of nonsingular solutions in the analytical model may be to satisfy det (T)=0, and when ϕ and α are given, γ may be obtained by solution.

As a preferable technical solution of the present disclosure, periodic solution may include solving formulas

$$\frac{2 {v}^{2}}{κ} ( {β}_{k}^{n} - k ) {( {β}_{k}^{n} + k )}^{2} \frac{{β}_{k}^{n}}{k} {h}_{k}^{n} + {\sum }_{j = - ∞}^{∞} {c}_{k - j} {h}_{j}^{n} = i τ {\sum }_{j = - ∞}^{∞} {c}_{k - j} ( {h}_{j}^{n - 1} - {h}_{j}^{n + 1} ) \\ and {\sum }_{j = - ∞}^{∞} {c}_{k - j} {h}_{j}^{n} = i τ {\sum }_{j = - ∞}^{∞} {c}_{k - j} ( {h}_{j}^{n - 1} - {h}_{j}^{n + 1} )$$

to obtain τ and corresponding eigenvectors; and on this basis, according to the following formula:

$$h ( x , t ) = {e}^{γ t} {\sum }_{n = - ∞}^{∞} {\sum }_{k = - ∞}^{∞} {h}_{k}^{n} ( y ) {e}^{i n t} {e}^{ikx} .$$

the periodic solution h(x,t) may be solved.

The present disclosure is compact in structure and simple in operation. By vertically rotating a short beam by 90° around an optical axis, a table board installed on a fixed seat can be placed vertically, avoiding the disassembly, assembly, and storage of the table board, which is conducive to saving the interior space of a recreational vehicle (RV), and has strong practicability and application value.

### BRIEF DESCRIPTION OF THE DRAWINGS

FIG. 1 is a two-dimensional cochlear model diagram of a preferred embodiment of the present disclosure;

FIG. 2 is a diagram of a real part solution and an imaginary part solution of γ satisfying det(T)=0 in the preferred embodiment of the present disclosure;

FIG. 3 is a dimensionless displacement amplitude graph of the BM in the preferred embodiment of the present disclosure;

FIG. 4 is a diagram of variation of a peak position of vibration of the BM with a frequency in the preferred embodiment of the present disclosure;

FIG. 5 is a graph of variation of displacement of the BM with time in the preferred embodiment of the present disclosure;

FIG. 6 is a graph of variation of displacement of the BM with time in the preferred embodiment of the present disclosure (where ω=400 s⁻¹, τ=0.05, and 0.08 and 0.1 correspond to upper, middle, and lower respectively);

FIG. 7 is a graph of variation of displacement of the BM with time in the preferred embodiment of the present disclosure (where ω=600 s⁻¹, and τ=0.05, 0.08, and 0.1 corresponds to upper, middle and lower respectively);

FIG. 8 is a graph of variation of displacement of the BM with time in the preferred embodiment of the present disclosure (where ω=800 s⁻¹, and τ=0.05, 0.08, and 0.1 corresponds to upper, middle and lower respectively);

FIGS. 9A-E are graphs of variation of displacement of the BM with positions at different frequencies in the preferred embodiment of the present disclosure; and

FIG. 10 shows a schematic block diagram of a computer that can be used for implementing the method and the system according to the embodiments of the present disclosure.

### DETAILED DESCRIPTION OF THE EMBODIMENTS

It should be noted that embodiments and features in the embodiments may be combined with each other without conflict, and the technical solutions of embodiments of the present disclosure are clearly and completely described below with reference to the accompanying drawings. Apparently, the described embodiments are merely some rather than all of the embodiments of the present disclosure. Based on the embodiments of the present disclosure, all other embodiments obtained by those of ordinary skill in the art without creative efforts shall fall within the protection scope of the present disclosure.

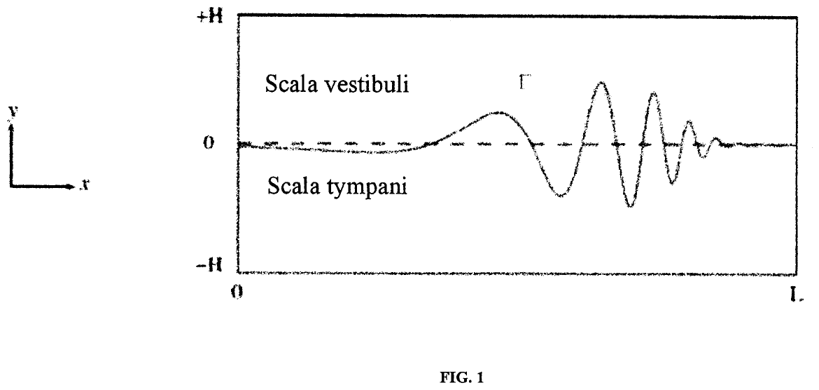

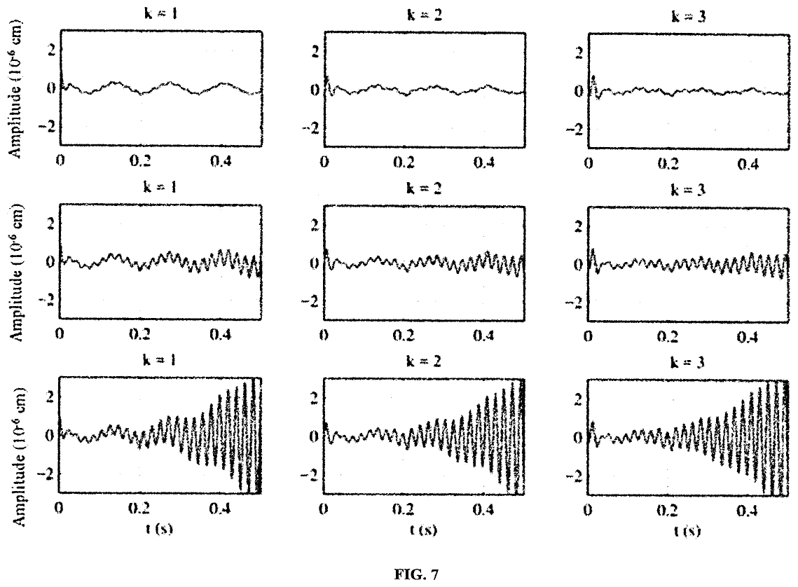

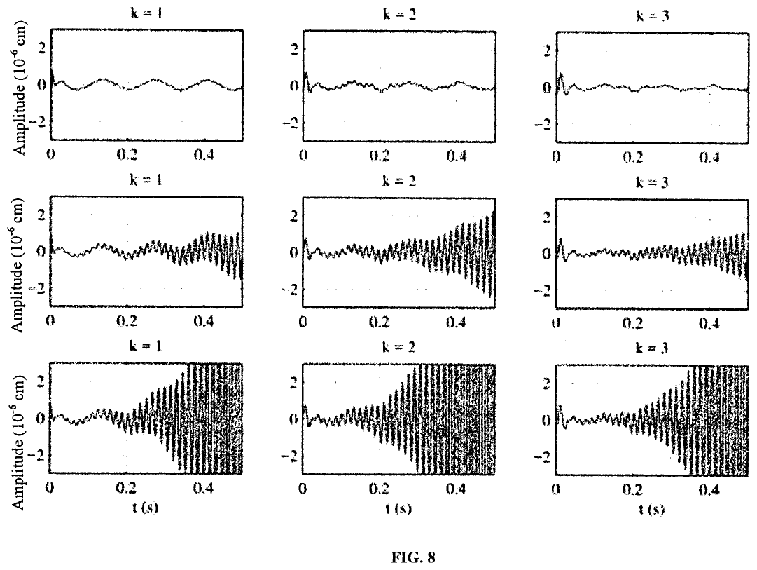

Referring to FIG. 1 to FIG. 9, a preferred embodiment of the present disclosure provides a computational method for considering contribution of biological activity to a cochlear sensory amplification mechanism.

The present disclosure mainly analyzes vibration characteristics of the BM of a sensory structure in a cochlear system, so the cochlear model is reasonably simplified, and considers a simplified 2 dimensional cochlear model, as shown in FIG. 1. A cochlear channel is a rectangular structure with two chambers, the length of which is L, and the height of which is H, and the BM is located in the center of the chamber. The influence of the essential characteristics of the BM on the vibration is studied. In FIG. 1, the solid line represents the vibration of BM away from the equilibrium position, the dashed line represents the equilibrium position of BM, the length direction along the cochlea is defined as the x-axis, and the thickness direction is the y-axis.

Based on the incompressible N-S equation, there are:

$$ρ ( \frac{\partial u}{\partial t} + u \cdot ∇ u ) = - ∇ p + μΔ u + f \\ ∇ \cdot u = 0. \qquad ( 1 )$$

In the formula, u(x,t) represents the fluid velocity, p(x,t) represents the pressure, ρ represents the density, and μ represents the viscosity coefficient. The present disclosure mainly studies the variation of stiffness in time and space, then the volume force f is:

$$f ( x , t ) = {\int }_{0}^{L} K ( s , t ) ( {X}_{0} - X ) δ ( x - X ) ds . \qquad ( 2 )$$

In the formula, δ(x) represents a two-dimensional Dirac Delta function, X(s,t) represents position parameters of the BM in a Lagrangian coordinate, and X₀(s)=(s, 0) represents an equilibrium position. The volume force f of the equation can be regarded to be determined by the intrinsic property stiffness K(s, t) of the BM. The vibration characteristics of the BM under the adjustment of intrinsic properties can be described by the variation of the parameters of the BM stiffness. A stiffness parameter of the BM is assumed to vary with time and space, and is expressed by a function as:

$$K ( s , t ) = σ {e}^{- λ s} ( 1 + 2 τ sin ( ω t ) ) . \qquad ( 3 )$$

In the formula, σ is an elastic stiffness constant in a mean time interval, λ describes a variation of stiffness along a BM space, and from previous experimental data, the stiffness of the BM is found to present an exponential variation rule along a length; an exponential function is used to describe the spatial variation of the stiffness. In addition, a periodic variation rule of the stiffness in a periodic vibration process of the BM is described by an amplitude parameter τ and a frequency ω.

According to coupled vibration interface conditions between fluid and the BM, the following is derived:

$$\frac{\partial X}{\partial t} = u ( X , t ) = {\int }_{Ω} u ( x , t ) δ ( x - X ) dx . \qquad ( 4 )$$

In order to make the present disclosure universal, dimensionless processing is used, and a dimensionless quantity is as follows:

$$x = \frac{L \tilde{x}}{π} , \\ t = \frac{\tilde{t}}{ω} , \\ u = {U}_{c} \tilde{u} , \\ p = {P}_{c} \tilde{p} , \\ X = \frac{L \tilde{X}}{π} , \\ s = \frac{L \tilde{s}}{π} . \qquad ( 5 )$$

In the formula, a subject on a wavy line is the dimensionless quantity. Uc and Pc respectively represent characteristic scales of velocity and pressure. By substituting the above dimensionless quantity into the formula (4), the following is obtained:

$$\frac{\partial \tilde{u}}{\partial \tilde{t}} + \tilde{u} \cdot \tilde{∇} \tilde{u} = - \tilde{∇} \tilde{p} + v \tilde{Δ} \tilde{u} + \tilde{f} \qquad ( 6 )$$

$$\tilde{∇} \cdot \tilde{u} = 0 \qquad ( 7 )$$

$$\tilde{f} ( \tilde{x} , \tilde{t} ) = {\int }_{0}^{π} \tilde{K} ( {\tilde{X}}_{0} - \tilde{X} ) \overline{δ} ( \tilde{x} - \tilde{X} ) d \tilde{s} . \qquad ( 8 )$$

$$\tilde{K} ( \tilde{s} , \tilde{t} ) = κ {e}^{- α \tilde{s}} ( 1 + 2 τ sin \tilde{t} ) \qquad ( 9 )$$

$$\frac{\partial \tilde{X}}{\partial \tilde{t}} = ( \tilde{X} , \tilde{t} ) \qquad ( 10 )$$

The characteristic scales of velocity and pressure are expressed as:

$${U}_{c} = \frac{L ω}{π} , \\ {P}_{c} = \frac{ρ {ω}^{2} {L}^{2}}{{π}^{2}} . \qquad ( 11 )$$

Therefore, parameters in equations (6) and (9) are expressed as:

$$v = \frac{μ {π}^{2}}{ρ {L}^{2} ω} , \\ κ = \frac{σ π}{ρ L {ω}^{2}} , \\ α = \frac{λ L}{π} . \qquad ( 12 )$$

In order to solve and analyze the above equations, further processing is needed. As the amplitude of the BM is generally at the nanoscale, which is very small compared to the size of the cochlea channel and implies that the Reynolds number of the fluid is very small. Thus, the nonlinear effect of the fluid can be ignored. In addition, the vibrations of BM are mainly the lateral Y-direction vibrations, and this model only considers the Y-direction vibrations. In summary, the system equation can be simplified as:

$$\frac{\partial u}{\partial t} = - ∇ p + v Δ u . \qquad ( 13 )$$

$$∇ \cdot u = 0 \qquad ( 14 )$$

In addition, there are the following conditions:

$$p = - κ {e}^{- α x} ( 1 + 2 τ sin t ) h ( x , t ) u ( x , 0 , t ) = 0. v ( x , 0 , t ) = \frac{\partial h}{\partial t} \qquad ( 15 )$$

h(x,t) represents vertical displacement of the BM, p(x,t)=p(x,0⁺,t)−p(x,0⁻¹,t) represents a pressure difference of the BM, and u(x,y,t) and v(x,y,t) represent vertical and vertical velocities respectively.

Since there are time-varying stiffness parameters in the system, the solution of the system satisfies the following form:

$$u ( x , t ) = {e}^{γ t} P ( x , t ) . \qquad ( 16 )$$

In the formula, the function P(x,t) represents a periodic function with a period of 2π. An exponential factor determines the stability of the solution when t−>∞. Series representation of P(x,t) is performed in an interval of [−π,π] to obtain:

$$u ( x , y , t ) = {e}^{γ t} {\sum }_{n = - ∞}^{∞} {\sum }_{k = - ∞}^{∞} {u}_{k}^{n} ( y ) {e}^{i n t} {e}^{ikx} \qquad ( 17 )$$

$$v ( x , y , t ) = {e}^{γ t} {\sum }_{n = - ∞}^{∞} {\sum }_{k = - ∞}^{∞} {v}_{k}^{n} ( y ) {e}^{i n t} {e}^{ikx} . \qquad ( 18 )$$

$$p ( x , y , t ) = {e}^{γ t} {\sum }_{n = - ∞}^{∞} {\sum }_{k = - ∞}^{∞} {p}_{k}^{n} ( y ) {e}^{i n t} {e}^{ikx} \qquad ( 19 )$$

$$h ( x , t ) = {e}^{γ t} {\sum }_{n = - ∞}^{∞} {\sum }_{k = - ∞}^{∞} {h}_{k}^{n} ( y ) {e}^{i n t} {e}^{ikx} \qquad ( 20 )$$

In the formula, Fourier series expansion in space and time is performed on P(x,t). Parameters to be solved in the above equation are Fourier coefficients uₖⁿ, vₖⁿ, and pₖⁿ varying along the Y-axis. Equations (17-19) are substituted into equations (13) and (14) to obtain the poisson equation of pressure expressed as:

$$Δ p = {\sum }_{n , k = - ∞}^{∞} ( - {k}^{2} {p}_{k}^{n} ( y ) + {p}_{k}^{{n}^{″}} ( y ) ) {ε}_{k}^{n} = 0. \qquad ( 21 )$$

In the formula, εₖⁿ(x,t)=e^{[(γ+in)t+ikx]}, due to linear independence of εₖⁿ(x,t), there is:

$$- {k}^{2} {p}_{k}^{n} ( y ) + {p}_{k}^{{n}^{″}} ( y ) = 0. \qquad ( 22 )$$

By solving equation (22), the following is obtained:

$${p}_{k}^{n} ( y ) = \{ \begin{matrix}{α}_{k}^{n} {e}^{ky} , & y < 0 \\ {b}_{k}^{n} {e}^{- ky} , & y > 0\end{matrix} . \qquad ( 23 )$$

Equation (18) is substituted into equation (13), and there is:

$${\sum }_{n , k = - ∞}^{∞} ( γ + i n ) {v}_{k}^{n} ( y ) {ε}_{k}^{n} = {\sum }_{n , k = - ∞}^{∞} ( - {p}_{k}^{{n}^{′}} ( y ) - {vk}^{2} {v}_{k}^{n} ( y ) + ν {v}_{k}^{{n}^{″}} ( y ) ) {ε}_{k}^{n} . \qquad ( 24 )$$

After further derivation, an ordinary differential equation is obtained as follows:

$${v}_{k}^{{n}^{″}} ( y ) - {( {β}_{k}^{n} )}^{2} {v}_{k}^{n} ( y ) = \frac{1}{v} {p}_{k}^{{n}^{′}} ( y ) . \qquad ( 25 )$$

In the formula

$${β}_{k}^{n} = \sqrt{\frac{γ + i n}{V} + {k}^{2}} . \qquad ( 26 )$$

Supposing γ+in≠0 and k≠0, then a solution of formula (25) is:

$${v}_{k}^{n} ( y ) = \frac{1}{2 v {β}_{k}^{n}} \{ \begin{matrix}- \frac{2 k {β}_{k}^{n}}{{( {β}_{k}^{n} )}^{2} - {k}^{2}} {α}_{k}^{n} {e}^{ky} + ( \frac{k}{{β}_{k}^{n} - k} {α}_{k}^{y} + \frac{k}{{β}_{k}^{n} + k} {b}_{k}^{y} ) {e}^{{β}_{k}^{n} y} , y < 0 \\ \frac{2 {β}_{k}^{n}}{{( {β}_{k}^{n} )}^{2} - {k}^{2}} {b}_{k}^{n} {e}^{- ky} + ( \frac{k}{{β}_{k}^{n} - k} {b}_{k}^{y} + \frac{k}{{β}_{k}^{n} + k} {α}_{k}^{y} ) {e}^{- {β}_{k}^{n} y} , y > 0\end{matrix} . \qquad ( 27 )$$

According to continuity, an equation is obtained:

$$i k {u}_{k}^{n} ( y ) + {v}_{k}^{{n}^{′}} ( y ) = 0. \qquad ( 28 )$$

Then the equation is solved to obtain:

$${u}_{k}^{n} ( y ) = \frac{- i}{2 k v} \{ \begin{matrix}- \frac{2 {k}^{2}}{{( {β}_{k}^{n} )}^{2} - {k}^{2}} {α}_{k}^{n} {e}^{ky} + ( \frac{k}{{β}_{k}^{n} - k} {α}_{k}^{y} + \frac{k}{{β}_{k}^{n} + k} {b}_{k}^{y} ) {e}^{{β}_{k}^{n} y} , y < 0 \\ - \frac{2 k}{{( {β}_{k}^{n} )}^{2} - {k}^{2}} {b}_{k}^{n} {e}^{- ky} + ( \frac{k}{{β}_{k}^{n} - k} {b}_{k}^{y} + \frac{k}{{β}_{k}^{n} + k} {α}_{k}^{y} ) {e}^{- {β}_{k}^{n} y} , y > 0\end{matrix} . \qquad ( 29 )$$

According to the continuity, interface boundary conditions are derived:

$${u}_{k}^{n} ( {0}^{+} ) = {u}_{k}^{n} ( {0}^{-} ) = \frac{i}{2 v} ( \frac{1}{{β}_{k}^{n} + k} {α}_{k}^{n} + \frac{1}{{β}_{k}^{n} + k} {b}_{k}^{n} ) = 0 \qquad ( 30 )$$

$${v}_{k}^{n} ( {0}^{+} ) = {v}_{k}^{n} ( {0}^{-} ) = \frac{1}{2 v {β}_{k}^{n}} ( \frac{- k}{{β}_{k}^{n} + k} {α}_{k}^{n} + \frac{k}{{β}_{k}^{n} + k} {b}_{k}^{n} ) = ( γ + in ) {h}_{k}^{y} . \qquad ( 31 )$$

After further derivation, the following is obtained:

$${α}_{k}^{n} = - v ( γ + in ) \frac{{β}_{k}^{n} + k}{k} {β}_{k}^{n} {h}_{k}^{n} \qquad ( 32 )$$

$${b}_{k}^{n} = v ( γ + in ) \frac{{β}_{k}^{n} + k}{k} {β}_{k}^{n} {h}_{k}^{n} . \qquad ( 33 )$$

By substituting equations (32) and (33) into equation (23) and further into equation (13), the following is obtained:

$${\sum }_{n , k = - ∞}^{∞} 2 v ( γ + in ) \frac{{β}_{k}^{n} + k}{k} {β}_{k}^{n} {h}_{k}^{n} {ε}_{k}^{n} = {\sum }_{n , k = - ∞}^{∞} - κ {e}^{- α x} ( 1 + 2 τ sin t ) {h}_{k}^{n} {ε}_{k}^{n} . \qquad ( 34 )$$

When γ+in=0 and k=0, equation (34) is simplified as:

$$0 = {\sum }_{n , k = - ∞}^{∞} - κ {e}^{- α x} ( 1 + 2 τ sin t ) {h}_{k}^{n} {ε}_{k}^{n} . \qquad ( 35 )$$

After Fourier expansion of the exponential function and sine function in the above formula, there is 1+2τ sin t=1−iτe^{it}+iτe^{-it}.

Since e^{-ax} is not a periodic function in [0,π], its Fourier series cannot converge in [0,π]. Therefore, the function is extended to the interval of [−π,π], and only the interval of x≥0 is considered. Therefore, even function periodic expansion of the exponential function is performed, and when γ+in≠0 and k≠0, there is:

$${\sum }_{n , k = - ∞}^{∞} 2 v ( γ + in ) \frac{{β}_{k}^{n} + k}{k} {β}_{k}^{n} {h}_{k}^{n} {ε}_{k}^{n} = {\sum }_{n , k = - ∞}^{∞} - κ ( {\sum }_{j = - ∞}^{∞} {c}_{j} {e}^{ijx} ) ( 1 - i τ {e}^{it} + i τ {e}^{- i t} ) {h}_{k}^{n} {ε}_{k}^{n} . \qquad ( 36 )$$

When γ+in=0 and k=0, there is:

$$0 = {\sum }_{n , k = - ∞}^{∞} - κ ( {\sum }_{j = - ∞}^{∞} {c}_{j} {e}^{ijx} ) ( 1 - i τ {e}^{it} + i τ {e}^{- i t} ) {h}_{k}^{n} {ε}_{k}^{n} , \qquad ( 37 )$$

where

$${c}_{j} = α \frac{1 - {( - 1 )}^{j} {e}^{- α x}}{π ( {α}^{2} + {j}^{2} )} \qquad ( 38 )$$

is a Fourier coefficient of the exponential function. By sorting out coefficients of a term εₖⁿ, the following is obtained:

$$\frac{2 {v}^{2}}{κ} ( {β}_{k}^{n} - k ) {( {β}_{k}^{n} + k )}^{2} \frac{{β}_{k}^{n}}{k} {h}_{k}^{n} + {\sum }_{j = - ∞}^{∞} {c}_{k - j} {h}_{j}^{n} = i τ {\sum }_{j = - ∞}^{∞} {c}_{k - j} ( {h}_{j}^{n - 1} - {h}_{j}^{n + 1} ) . \qquad ( 39 )$$

When γ+in=0 and k=0, there is:

$${\sum }_{j = - ∞}^{∞} {c}_{k - j} {h}_{j}^{n} = i τ {\sum }_{j = - ∞}^{∞} {c}_{k - j} ( {h}_{j}^{n - 1} - {h}_{j}^{n + 1} ) . \qquad ( 40 )$$

When α≠0, the above equations (39) and (40) constitute a linear system, in which hⱼⁿ is a stiffness parameter of spatial variation and the coupling effect between various spatial modes is considered.

Since the present disclosure mainly studies the stability of a cochlea mechanical system, we mainly solve periodic solutions in equations (39) and (40), that is, when Re{γ}=0. In addition, for the solution of the stability boundary, there are two kinds of values of the parameters γ. The first one is γ=0, which corresponds to the harmonic solution of the system. The second one is γ=½*i, which corresponds to the sub-harmonic solution of the system. In order to ensure that the value of h(x,t) is in the real number range, a condition hₖⁿ=h̅₋ₖ⁻ⁿ is introduced where h with a line across the head represents the complex conjugate of h. In order to ensure space symmetry of even functions of solutions, there are:

$${h}_{- k}^{n} = {h}_{k}^{n} \qquad ( 41 )$$

$${h}_{- k}^{n} = \{ \begin{matrix}{\overline{h}}_{k}^{n} , & γ = 0 \\ {\overline{h}}_{k}^{n - 1} , & γ = \frac{1}{2} i\end{matrix} . \qquad ( 42 )$$

For the solution (16), the following periodic conditions are implied:

$$u ( x , t + 2 π n ) = {e}^{γ ( t + 2 π n )} P ( x , t ) = {ξ}^{n} u ( x , t ) . \qquad ( 43 )$$

For any positive integer n, when ξ=e^{γ2π} and t takes a fixed value, the above conditions are satisfied. When n−>∞, if |ξ|<1, the solution will be stable. If |ξ|>1, the solution will be non-stable. When ξ=±1, it corresponds to the periodic boundary condition of the periodic solutions from stable to unstable state. If γ=0, then ξ=1, and there is:

$$u ( x , t + 2 π ) = u ( x , t ) . \qquad ( 44 )$$

The above formula is a harmonic solution with a period of 2π. If γ=½*i, then ξ=−1, and there is:

$$u ( x , t + 2 π ) = - u ( x , t ) u ( x , t + 4 π ) = u ( x , t ) . \qquad ( 45 )$$

The above formula is a doubly periodic sub-harmonic solution. In order to solve equations (39) to (42), the system equation is reduced, n=0, 1, . . . , N, and k=1, 2, . . . , M, and by matrix representation, the following is obtained:

$$A \vec{h} = τ B \vec{h} , where \qquad ( 46 )$$

$$\vec{h} = {[ … , Re ( {h}_{k}^{n} ) , Im ( {h}_{k}^{n} ) , Re ( {h}_{k - 1}^{n} ) , Im ( {h}_{k + 1}^{n} ) , … ]}^{T} . \qquad ( 47 )$$

The above equations include 2*M*(N+1) unknown coefficients to be solved, and A and B are skew diagonal matrices. The skew diagonal matrix A is expressed as A=diag(A⁰, A¹, . . . , A^{N}) and has the following forms:

$${A}^{n} = [ \begin{matrix}{C}_{1 , 1} + {D}_{1}^{n} & {C}_{1 , 2} & … & {C}_{1 , M} \\ {C}_{2 , 1} & {C}_{2 , 2} + {D}_{2}^{n} & … & {C}_{2 , M} \\ ⋮ & ⋮ & ⋱ & ⋮ \\ {C}_{M , 1} & {C}_{M , 2} & … & {C}_{M , M} + {D}_{M}^{n}\end{matrix} ] , where \qquad ( 48 )$$

$${C}_{k , j} = [ \begin{matrix}{C}_{k - j} + {c}_{k + j} & 0 \\ 0 & {c}_{k - j} + {c}_{k + j}\end{matrix} ] , \qquad ( 49 )$$

and

$${D}_{k}^{n} = \frac{2 {v}^{2}}{κ k} [ \begin{matrix}Re \{ ( {β}_{k}^{n} - k ) {( {β}_{k}^{n} + k )}^{2} {β}_{k}^{n} \} & - Im \{ ( {β}_{k}^{n} - k ) {( {β}_{k}^{n} + k )}^{2} {β}_{k}^{n} \} \\ Im \{ ( {β}_{k}^{n} - k ) {( {β}_{k}^{n} + k )}^{2} {β}_{k}^{n} \} & Re \{ ( {β}_{k}^{n} - k ) {( {β}_{k}^{n} + k )}^{2} {β}_{k}^{n} \}\end{matrix} ] . \qquad ( 50 )$$

The triangular skew diagonal matrix B has the following forms:

$$B = [ \begin{matrix}\hat{B} & \hat{B} & & & \\ \hat{B} & 0 & \hat{B} & & \\ & ⋱ & ⋱ & ⋱ & \\ & & \hat{B} & 0 & \hat{B} \\ & & & \hat{B} & 0\end{matrix} ] , where \qquad ( 51 )$$

$$\hat{B} = [ \begin{matrix}{\hat{C}}_{1 , 1} & {\hat{C}}_{1 , 2} & … & {\hat{C}}_{1 , M} \\ {\hat{C}}_{2 , 1} & {\hat{C}}_{2 , 1} & & {\hat{C}}_{2 , M} \\ ⋮ & & ⋱ & ⋮ \\ {\hat{C}}_{M , 1} & {\hat{C}}_{M , 2} & … & {\hat{C}}_{M , M}\end{matrix} ] , \qquad ( 52 )$$

$${\hat{C}}_{k , j} = [ \begin{matrix}0 & - {c}_{k - j} - {c}_{k + j} \\ {c}_{k - j} + {c}_{k + j} & 0\end{matrix} ] .$$

Both A and B matrices are known, and the solution of equation (46) can be regarded as an eigenvalue problem, that is:

$${A}^{- 1} B \vec{h} = \frac{1}{τ} \vec{h} . \qquad ( 53 )$$

An eigenvalue of the stability solution in the above equation is 1/τ. For the physical problems discussed herein, only those values whose τ is a real number and less than ½ are focused on, so as to ensure that the stiffness function K (s, t) is real and non-negative.

When τ=0 in the stiffness function of the BM, the stiffness function is a non-periodic function, the solution of the equation is stable, and the Fourier coefficient of the solution satisfies:

$$\frac{2 ϕ γ}{k v} \sqrt{\frac{γ}{v} + {k}^{2}} ( k + \sqrt{\frac{γ}{v} + {k}^{2}} ) {h}_{k}^{0} + {\sum }_{j = - ∞}^{∞} {c}_{k - j} {h}_{j}^{0} = 0. \qquad ( 54 )$$

When k=0, there is:

$${\sum }_{j = - ∞}^{∞} {c}_{k - j} {h}_{j}^{0} = 0. \qquad ( 55 )$$

In formula (54), ϕ=ν²/κ=π³μ²/(ρσL³) represents a ratio of a fluid viscosity resistance to an elastic force of the BM. The above equation can be simply expressed as T\vec{h}⁰=0, and the T matrix depends on parameters ϕ, γ, and α. A condition for existence of nonsingular solutions in the system is to satisfy det (T)=0, and when ϕ and α are given, γ is obtained by solution.

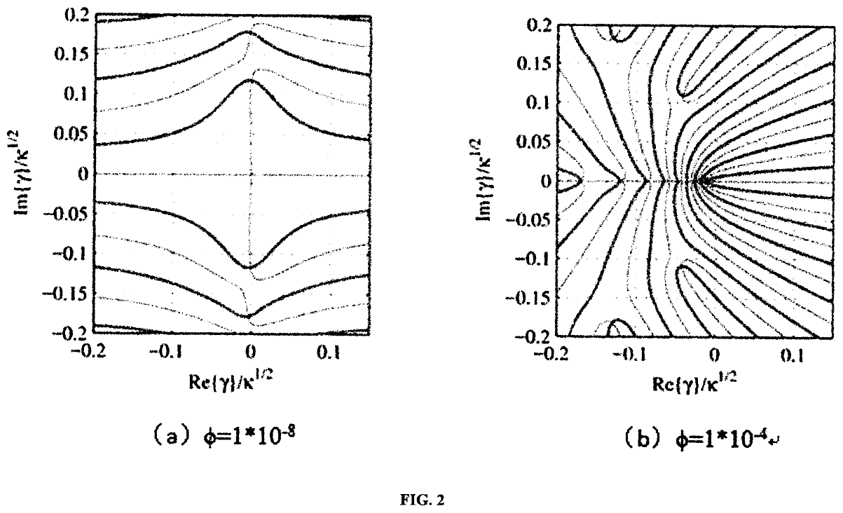

When α=1, and M=20, γ of two cases ϕ=1*10⁻⁸ and 1*10⁻⁴ are calculated respectively. The results are shown in FIG. 2. The thick line represents the real part solution and the narrow line represents the imaginary part solution. The cross point of the real part and the imaginary part corresponds to the resonant mode of the system.

It can be seen from FIG. 2 that the cross point of the real part and the imaginary part is always negative, which means that the solution is stable. For ϕ that is quite small, the main modes of Im{γ} are nonzero, so the solutions are oscillatory. When ϕ increases to 1*10⁻⁴, the influence of fluid viscosity increases, and the main mode will exhibit attenuation and no oscillation. In the above both cases, the system mode attenuates with the increase of time. Therefore, when τ=0, the periodic solution or unstable solution does not exist, and the non-zero τ parameter of time-varying effect must be considered in the stiffness function before the periodic solution exists.

When τ≠0 is considered, the stiffness function of the BM is equivalent to introduction of an intrinsic mediation mechanism that varies with time. The effect of this mechanism is equivalent to considering the biological activity of the BM, i.e., the material of the BM varies with time and has self-repairing property. τ and corresponding eigenvectors can be obtained by solving formulas (39) and (40). On this basis, the periodic solution h(x,t) is solved with formula (20). The parameters selected in the calculation are shown in Table 1.

TABLE 1
Parameters of human cochlear system for calculation
Physical parameters

| | |
|---|---|
| Fluid density | ρ = 1.0 g cm⁻³ |
| Fluid viscosity coefficient | M = 0.02 g cm⁻¹ s⁻¹ |
| Elastic stiffness constant | σ = 6*10⁵ g cm⁻² s⁻² |
| of BM | |
| Spatial variation coefficient | λ = 1.4 cm⁻¹ |
| of elastic stiffness | |
| Length of BM | L = 3.5 cm |
| Stimulus frequency | ω∈[400, 5000] s⁻¹ |
| Characteristic scale of | Uc = ωL/π∈[446, 5573] cm s⁻¹ |
| velocity | |
| Characteristic scale of | Pc = ρω²L²/π²∈[2*10⁵, 3.1*10⁷] dyn cm⁻² |
| pressure | |

Non-dimensional parameters

| | |
|---|---|
| Dimensionless attenuation | α = 1 |
| rate | |
| Periodic amplitude of | τ = ∈[0, 0.5] |
| stiffness | |
| Dimensionless viscosity | υ∈[8.06*10⁻⁶, 1.61*10⁻⁴] |
| Dimensionless stiffness | κ∈[0.135, 53.9] |
| Relative gravity of | ϕ = υ²/κ = π³μ²/(ρσL³) = 4.8* 10⁻¹⁰ |
| viscosity and stiffness | |

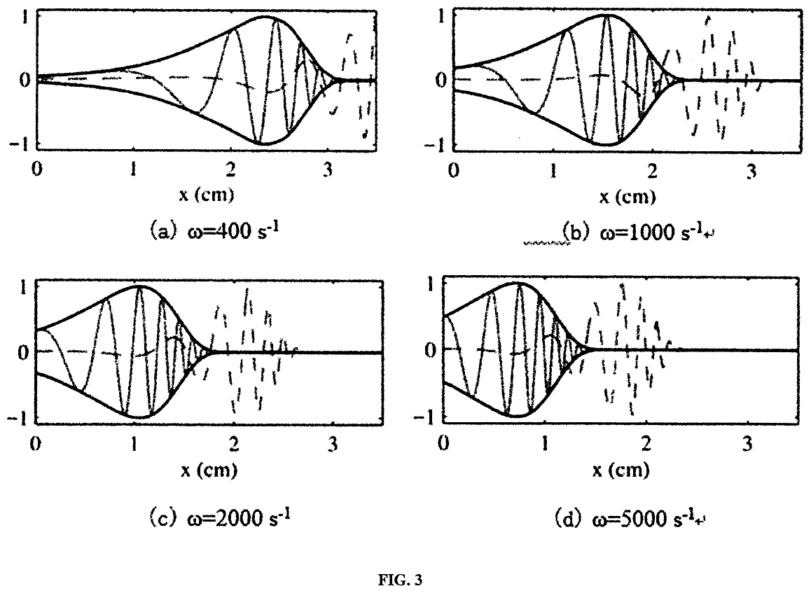

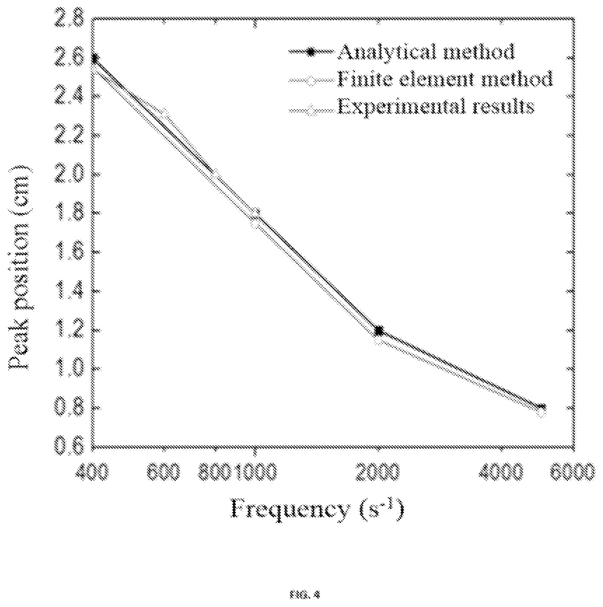

According to the parameters in Table 1, vertical displacement curves h (x,tₚₑₐₖ) of the BM at frequency ω=400, 1,000, 2,000, and 5,000 are calculated respectively. tₚₑₐₖ represents time corresponding to the maximum vertical displacement. The calculation results are shown in FIG. 3. The result of the curve is dimensionless. The envelope is determined by calculating the complex function of the BM. The real part of the complex function is the vibration amplitude of the BM, and the imaginary part is its Hilber transform. The inner wavy solid line represents harmonic vibration of the BM, the dashed line represents sub-harmonic vibration of the BM, and the outer wavy solid line represents the envelope curve of vibration of the BM.

The present disclosure is simulated using the COMSOL software.

Since the perilympha has low viscosity and is incompressible, and at the same time, the Reynolds number is low, a laminar flow model is selected as the fluid, and it is assumed to be incompressible viscous fluid. The N-S equation of the fluid is:

$$ρ \frac{\partial {u}_{fluid}}{\partial t} + ρ ( {u}_{fluid} \cdot ∇ ) {u}_{fluid} = ∇ \cdot [ - p + μ ( ∇ {u}_{fluid} + {( ∇ {u}_{fluid} )}^{T} ) ] + F . \qquad ( 56 )$$

$$ρ ∇ \cdot {u}_{fluid} = 0. \qquad ( 57 )$$

In the formula, ρ and μ represent density and viscosity coefficients of the fluid respectively, u_{fluid} and p represent the velocity and pressure of the fluid respectively, and F represents the density of the volume force acting on the fluid.

An interface between the BM and the perilympha is a fluid-structure interface, and there are:

$${u}_{fluid} = {u}_{solid} \qquad ( 58 )$$

$$σ \cdot n = Γ \cdot n , Γ = [ - p + μ ( ∇ {u}_{fluid} + {( ∇ {u}_{fluid} )}^{T} ) ] .$$

That is, the displacement of the solid on the interface is equal to the displacement of the fluid, the pressure generated by the fluid motion acts on the solid, and at the same time, the stress generated by the solid deformation reacts on the fluid, and solid element nodes and fluid element nodes on the interface are consistent and correspond to each other, and different from each other.

It can be seen from FIG. 3 that the envelope curve of vibration of the BM presents asymmetric distribution characteristics. From the base to the top, the amplitude of the curve increases slowly at first, and then decreases rapidly after reaching a peak point. The corresponding peak positions are different at different frequencies. It can be seen that the traveling wave vibration of the BM does not necessarily need to be realized by an external active force. Since the stiffness of the BM itself varies with time and space, the calculation results consistent with the previous pure-tone excitation model can also be obtained.

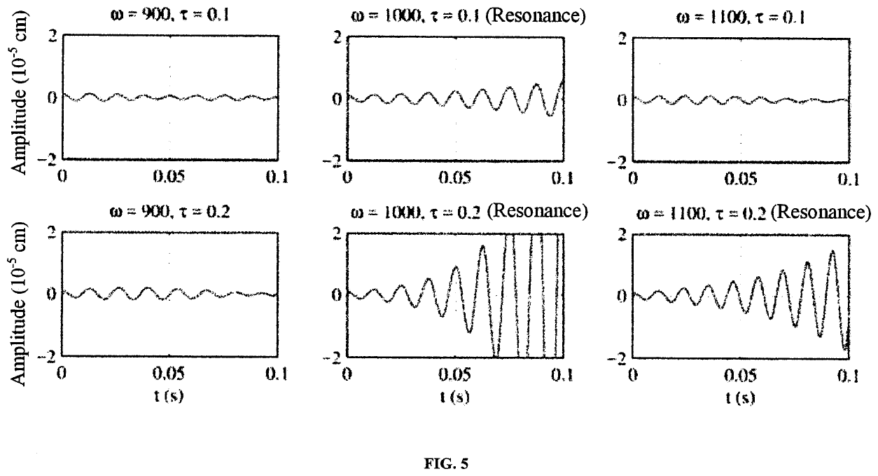

FIG. 4 calculates a relationship between a peak position of the curve shown in FIG. 3 and a frequency, and displays it in logarithmic coordinates. The solid square is the analytical solution calculated above, the hollow circle is the finite element simulation result, and the hollow triangle is the experimental test result. It can be seen from the figure that the peak position of vibration decreases linearly with the increase of frequency, and the point with frequency of 2000 s⁻¹ is a piecewise point. The analytical results are close to the numerical simulation results and the experimental results, so the analytical model provided in the present disclosure is reasonable and accurate.

In order to solve equation (53), first, a simple special case α=0 is considered, that is, the stiffness function of the BM does not depend on the position of the BM. Therefore, the Fourier series can be decoupled in space. For each spatial wave number k, there is:

$$\frac{2 ϕ}{k} ( {β}_{k}^{n} - k ) {( {β}_{k}^{n} + k )}^{2} {β}_{k}^{n} {h}_{k}^{n} + {h}_{k}^{n} = i τ ( {h}_{k}^{n - 1} - {h}_{k}^{n + 1} ) . \qquad ( 56 )$$

Formula (56) can be simply expressed as:

$${A}_{k} {\vec{h}}_{k} = τ {B}_{k} {\vec{h}}_{k} . \qquad ( 57 )$$

For each k, there are:

$${\vec{h}}_{k} = {[ Re ( {h}_{k}^{0} ) , Im ( {h}_{k}^{0} ) , … , Re ( {h}_{k}^{N} ) , Im ( {h}_{k}^{N} ) ]}^{T} , and \qquad ( 58 )$$

$${A}_{k} = diag ( I + {D}_{k}^{0} , I + {D}_{k}^{1} , … , I + {D}_{k}^{N} ) . \qquad ( 59 )$$

Dₖ^{N} is determined by formula (50). The parameters ω=900, 1,000, and 1,100 s⁻¹ are selected, and τ=0.1 and 0.2. The other parameters are listed in Table 1. The amplitudes of the BM under different parameters are calculated, as shown in FIG. 4.

It can be seen from FIGS. 5 (α=0 and k=1) that when τ=0.1, for the cases when ω=900 s⁻¹ or 1,100 s⁻¹, vibration of the BM is stable. That is, with the increase of time, it is found that the BM is in near-equal vibration with a slight attenuation at the end of the moment. When ω=1,000 s⁻¹, with the increase of time, the amplitude of the BM increases gradually, and resonance occurs at the end of the moment. When τ=0.2, only when ω=900 s⁻¹, the BM vibration is stable. At other frequencies, resonance occurs in the BM. Compare the two situations, when ω=1,000 s⁻¹, resonance occurs in the BM, and the amplitude is the largest, while the amplitude at other frequencies is relatively small.

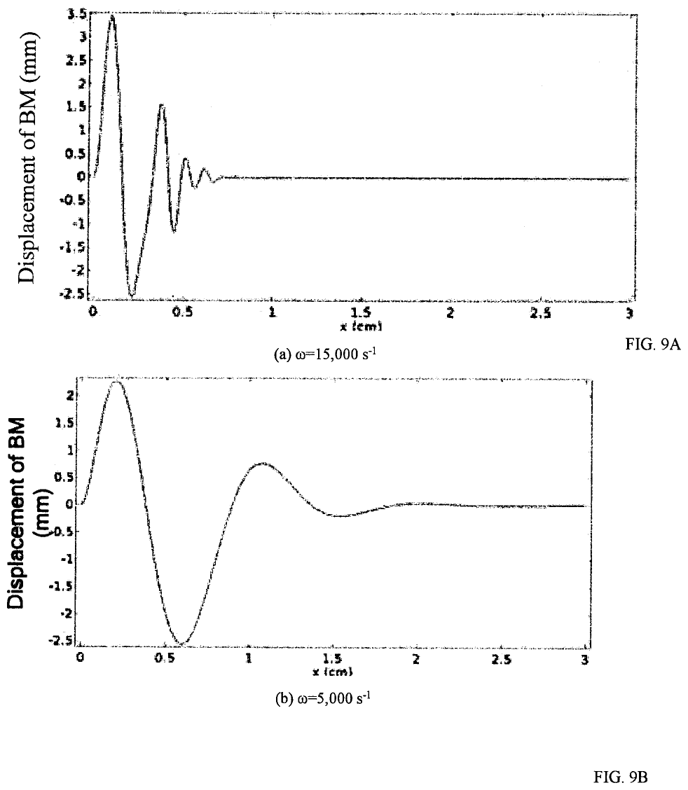

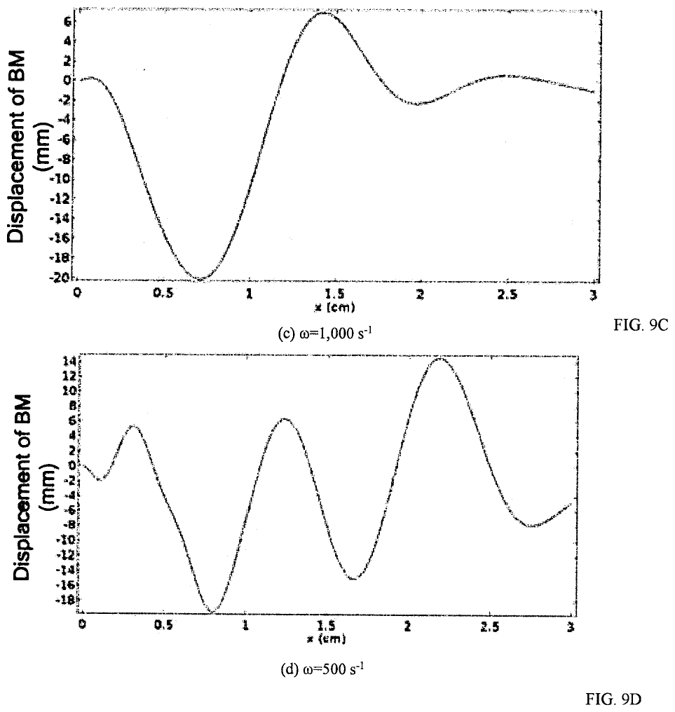

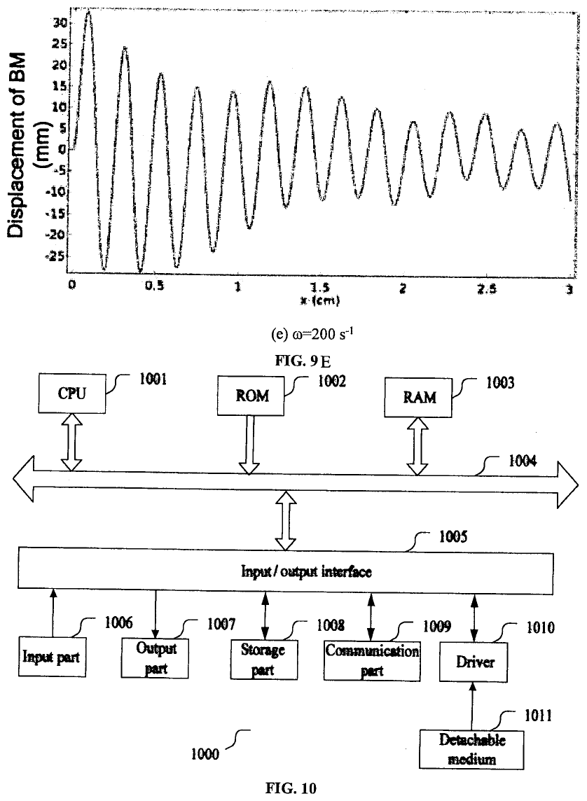

The finite element numerical simulation results established by the present disclosure are relatively consistent with the analytical model results (see FIG. 3, FIG. 4, and FIG. 9), and since the effect of the material of the BM varying with time is considered, it is jointly found that the BM has unstable resonant motion characteristics. The difference is that the analytical model does not consider external excitation, only the periodic variation modulation of the BM stiffness parameter causes an unstable overall resonance phenomenon in the BM, while the finite element model not only considers the time-varying properties of the material itself, and also considers the time-varying factor of external excitation, which means that only through the change of the biological activity of the BM itself, the BM can perceive sounds of different frequencies, thereby producing different vibration amplitudes and vibration forms.

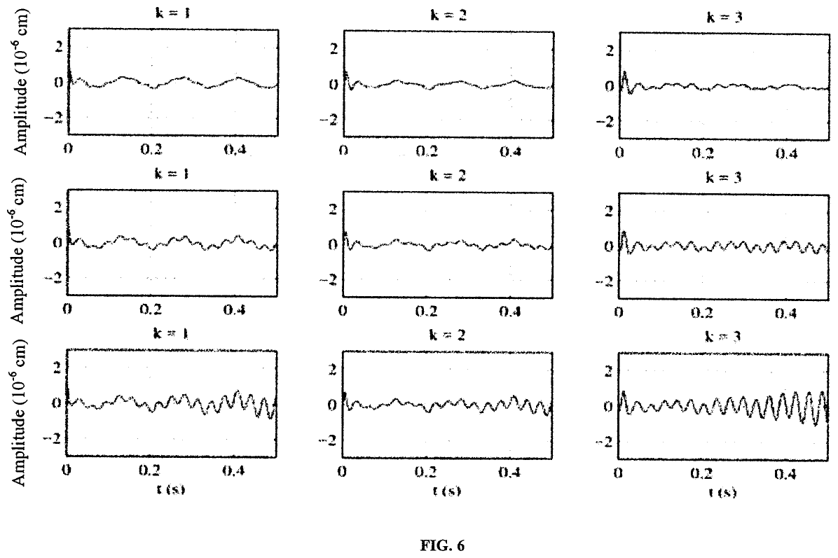

Then the case when α≠0 is continued to be analyzed, that is, the stiffness of the BM is coupled in the spatial position and varies exponentially with the variation of length. For the selected different parameters ω(400, 600, and 800 s⁻¹) and τ(0.05, 0.08, and 0.1), amplitude curves of variation of vibration of the BM with time are calculated, as shown in the FIG. 6 to FIG. 8.

It can be seen from the figures that when τ=0.05, the vibration of the BM attenuates gradually with the increase of time, but the change of an internal force caused by stiffness cannot significantly cause the instability of the vibration of the BM. the system mode attenuates with the increase of time. When τ is raised to 0.08, the instability of the vibration of the BM will be obviously enhanced, and periodic fluctuations will be maintained. When τ is raised to 0.1, the instability of the vibration of the BM will be very intense.

In summary, the time-varying effect of stiffness parameters is reflected in the frequency ω and the periodic amplitude parameter τ. With the variation of frequency and amplitude parameters, the BM will resonate violently, which can trigger the acute active auditory process and amplify the sound process of the cochlear. The resonance phenomenon of the BM will become more intense with the increase of periodic amplitude parameter τ, while the resonance phenomenon at low frequency is more significant than that at medium and high frequencies. The parameter variation of BM stiffness in space and time is due to the spatial distribution and biological activity of the materials of the BM structure itself, but not to the external feedback forces, that is, the vibration and sensory amplification process of the BM is realized by the intrinsic properties (biological activity) of the material of the BM.

Based on the established finite element model, a relationship of variation of the displacement of the BM in the Y-direction with space at different stimulus frequencies is calculated, as shown in FIG. A-3. In this figure, the high frequency 15,000 s⁻¹, the middle frequencies 5,000 s⁻¹ and 1,000 s⁻¹, and the low frequencies 500 s⁻¹ and 200 s⁻¹ are selected. It can be seen from the figure that when the stimulus frequency decreases from high frequencies to low frequencies, the maximum displacement of the BM propagates from the base to the top. However, when the frequency decreases to 500 s⁻¹, the vibration form of the BM is different from the traveling wave vibration (only local violent vibration). The BM not only has a local peak, but also resonates along the entire space. When the frequency continues to decrease to 200 s⁻¹, the BM as a whole produces a relatively intense resonance phenomenon, which explains the experimental phenomena that cannot be explained by the traveling wave theory, that is, the BM produces resonance at low frequency that is different from the traveling wave vibration.

Based on the established finite element model, a relationship of variation of the displacement of the BM in the Y-direction with space at different stimulus frequencies is calculated, as shown in FIG. A-3. In this figure, the high frequency 15,000 s⁻¹, the middle frequencies 5,000 s⁻¹ and 1,000 s⁻¹, and the low frequencies 500 s⁻¹ and 200 s⁻¹ are selected. It can be seen from the figure that when the stimulus frequency decreases from high frequencies to low frequencies, the maximum displacement of the BM propagates from the base to the top. However, when the frequency decreases to 500 s⁻¹, the vibration form of the BM is different from the traveling wave vibration (only local violent vibration). The BM not only has a local peak, but also resonates along the entire space. When the frequency continues to decrease to 200 s⁻¹, the BM as a whole produces a relatively intense resonance phenomenon, which explains the experimental phenomena that cannot be explained by the traveling wave theory, that is, the BM produces resonance at low frequency that is different from the traveling wave vibration.

In the present disclosure, a biomechanical model considering characteristics (biological activity) of the periodic variation of the stiffness parameters of the BM with time and space is established. It not only reproduces the vibration behavior of the BM traveling wave in the past test, but also verifies the correctness of the model. It is also found that there is an unstable resonance phenomenon in the BM. Through analysis and numerical simulation, the following conclusions can be drawn:

(1) When there is no time-varying effect on the stiffness of the BM, that is, when τ=0, the vibration of the BM is stable. Considering the viscosity of the fluid, the vibration of the BM gradually attenuates with the increase of time. When the BM has biological activity, that is, when τ≠0, the vibration of the BM becomes unstable and presents periodic vibration characteristics.

(2) When the frequency range is greater than or equal to 400 s⁻¹, the vibration of the BM presents the vibration characteristics of the traveling waves. With the increase of frequency parameters of the stiffness, the maximum amplitude of the BM gradually shifts from the base top to the base floor. The amplitude gradually decreases with the viscosity of the fluid and the damping of the BM itself. When the frequency is less than 400 s⁻¹, the vibration form of the BM is different from the traveling wave vibration (only local violent vibration). The BM not only has a local peak, but also resonates along the entire space. When the frequency is 200 s⁻¹, the BM as a whole produces a relatively intense resonance phenomenon, which explains the experimental phenomena that cannot be explained by the traveling wave theory^{[10,39-40],} that is, the BM produces resonance at low frequency that is different from the traveling wave vibration.

(3) When describing the spatial position variation parameter of the stiffness of the BM α=0, that is, the coupling between the time-varying parameter and the space-varying parameter is not considered. When ω=1,000 s⁻¹, no matter if the time parameter τ=0.1 or 0.2, unstable resonance phenomenon occurs in the BM, and the amplitudes at other frequencies are small. Only when the time parameter τ increases gradually, the stable resonance motion of the BM will occur at other frequencies.

(4) When α≠0, that is, the stiffness of the BM is coupled in the spatial position, the BM undergoes unstable vibration at different frequencies, and with the increase of τ, the unstable vibration of the BM becomes intense, accompanied by the resonance phenomenon.

Through the study of the present disclosure, an important conclusion is drawn: without considering the external excitation, modulating the BM stiffness parameters periodically in time and space will lead to unstable global resonance phenomenon of the BM, which will lead to sensitive perception and sound amplification process of the cochlea, implying that the strong coupling vibration between the BM and the surrounding lymph fluid can be produced only through the changes of the biological activity of the BM itself without an additional force (that is, part of the mechanism of sound amplification by the cochlea originates from the biological activity of the BM and its microstructure, which has not been reported in previous studies). The computational model can help humans to comprehensively reveal the amplification mechanism of the cochlear to sound.

An important conclusion can be deduced from the results of the present disclosure: applying the principles of physical mechanics to model and analyze living organisms should break through the classical theoretical methods of studying objects in the past, and supplement the changes of biological activities of living organisms, which is the essential difference between biomechanics and general mechanics.

The present disclosure provides an accurate, feasible and efficient computational method for modeling and analyzing living organisms by applying the principles of physical mechanics.

The analytical model involved in the computational method according to the embodiment takes the physical parameters of the human cochlear system shown in Table 1 as an input and the vertical displacement h (x,t) of the cochlear basement membrane as an output, and builds a relationship between physical parameters of the human cochlear system and the vertical displacement of the cochlear basement membrane. For example, as the elastic stiffness coefficient of the basement membrane is changed, the vertical displacement h (x,t) of the cochlear basement membrane varies accordingly, and vice versa. Therefore, the computational method according to the embodiment can be applied to determine a hardening degree of the cochlear basement membrane. Specifically, a hearing-impaired person can be subjected to a hearing test by professional inspection equipment to obtain decibel value of hearing loss of the hearing-impaired person, thereby determining the amplitude of the basement membrane of the hearing-impaired person. In the analytical model, only the vibration in Y direction is considered, thus the amplitude of the basement membrane is equal to the vertical displacement. The elastic stiffness coefficient of the basement membrane of the normal person can be obtained by the analytical model according to the embodiments, with the amplitude of the basement membrane of the normal person as an input of the model. Similarly, the elastic stiffness coefficient of the basement membrane of the hearing-impaired person can be obtained by the analytical model according to the embodiments, with the amplitude of the basement membrane of the hearing-impaired person as an input of the model. Therefore, the hardening degree of the cochlear basement membrane of the hearing-impaired person can be calculated according to the elastic stiffness coefficient of the basement membrane of the normal person and the elastic stiffness coefficient of the basement membrane of the hearing-impaired person.

The amplitude of the basement membrane of the hearing-impaired person can be determined by calculating a decreased value of decreased amplitude of basement membrane of the hearing-impaired person according to a conversion relationship between the amplitude of the basement membrane and the decibel value of hearing loss based on the value of hearing loss of the hearing-impaired person; and calculating the amplitude of the basement membrane of the hearing-impaired person according to the amplitude of the basement membrane of the normal person and the calculated value of the decreased amplitude of the basement membrane of the hearing-impaired person. Alternatively, the amplitude of the basement membrane of the hearing-impaired person can be determined by subtracting the decibel value of hearing loss of the hearing-impaired person from the decibel value of the hearing of the normal person to obtain the decibel value of the hearing of the hearing-impaired person; and calculating the amplitude of the basement membrane of the hearing-impaired person according to a conversion relationship between the amplitude of the basement membrane and the decibel value, based on the decibel value of the hearing of the hearing-impaired person is subjected as an input. The above determining methods are merely exemplary, and other method which can determining the amplitude of the basement membrane of the hearing-impaired person can also be adopted.

The hardening degree of the cochlear basement membrane of the hearing-impaired person with respect to the cochlear basement membrane of the normal person can be obtained by calculating a difference between the elastic stiffness coefficient of the hearing-impaired person and the elastic stiffness coefficient of the normal person, and dividing the difference by the elastic stiffness coefficient of the basement membrane of the normal person. Therefore, the computational method according to the embodiment can be applied to determine the hardening degree of the cochlear basement membrane, thereby assisting the doctor in determining whether the hearing-impaired person has the symptom of cochlear tissue hardening, and thus personalized clinical diagnosis can be implemented.

In addition, the analytical model according to the embodiment can also be used in some functional devices for researching and developing cochlear implants or enhancing hearing. For example, the analytical model is integrated into a cochlear equipment to enhance hearing, or integrated into a cochlear testing equipment to test the hearing level of the cochlear implants and the functional devices for enhancing hearing. Therefore, the method according to the embodiment can assist in manufacturing cochlear equipment and cochlear testing equipment.

In addition, the respective steps in the above method can be implemented by software, firmware, hardwire or a combination thereof. In case of implementation by software or firmware, programs constituting the software are installed from a storage medium or a network to a computer having a dedicated hardware structure; the computer, when installed with various programs, can implement various functions and the like.

FIG. 10 shows a schematic block diagram of a computer that can be used for implementing the method and the system according to the embodiments of the present disclosure.

In FIG. 10, a central processing unit (CPU) 1001 executes various processing according to a program stored in a read-only memory (ROM) 1002 or a program loaded from a storage part 808 to a random access memory (RAM) 1003. In the RAM 1003, data needed at the time of execution of various processing and the like by the CPU 1001 is also stored according to requirements. The CPU 1001, the ROM 1002 and the RAM 1003 are connected to each other via a bus 1004. An input/output interface 1005 is also connected to the bus 1004.

The following components are connected to the input/output interface 1005: an input part 1006 (including a keyboard, a mouse and the like); an output part 1007 (including a display, such as a Cathode Ray Tube (CRT), a Liquid Crystal Display (LCD) and the like, as well as a loudspeaker and the like); the storage part 1008 (including a hard disc and the like); and a communication part 1009 (including a network interface card such as an LAN card, a modem and so on). The communication part 1009 performs communication processing via a network such as the Internet. According to requirements, a driver 1010 may also be connected to the input/output interface 1005. A detachable medium 1011 such as a magnetic disc, an optical disc, a magnetic optical disc, a semiconductor memory and the like may be installed on the driver 1010 according to requirements, such that a computer program read therefrom is installed in the storage part 1008 according to requirements.

In the case of carrying out the foregoing series of processing by software, programs constituting the software are installed from a network such as the Internet or a storage medium such as the detachable medium 1011.

Those skilled in the art should appreciate that such a storage medium is not limited to the detachable medium 1011 storing therein a program and distributed separately from the apparatus to provide the program to a user as shown in FIG. 10. Examples of the detachable medium 1011 include a magnetic disc (including floppy disc (registered trademark)), a compact disc (including compact disc read-only memory (CD-ROM) and digital versatile disc (DVD), a magneto optical disc (including mini disc (MD)(registered trademark)), and a semiconductor memory. Or, the storage medium may be hard discs and the like included in the ROM 1002 and the storage part 1008 in which programs are stored, and are distributed concurrently with the apparatus including them to users.

The present disclosure further proposes a program product storing therein a machine-readable instruction code that, when read and executed by a machine, can implement the aforesaid method according to the embodiment of the present disclosure.

Correspondingly, a storage medium for carrying the program product storing the machine-readable instruction code is also included in the disclosure of the present disclosure. The storage medium includes but is not limited to a floppy disc, an optical disc, a magnetic optical disc, a memory card, a memory stick and the like.

For those skilled in the art, it is apparent that the present disclosure is not limited to the details of the foregoing exemplary embodiments, and that the present disclosure can be implemented in other specific forms without departing from the spirit or basic features of the present disclosure. The embodiments should be regarded as exemplary and non-limiting in every respect, and the scope of the present disclosure is defined by the appended claims rather than the above description. Therefore, all changes falling within the meaning and scope of equivalent elements of the claims should be included in the present disclosure. Any reference numeral in the claims should not be considered as limiting the involved claims.

In addition, it should be understood that although this specification is described in accordance with the implementations, not every implementation includes only an independent technical solution. Such a description is merely for the sake of clarity, and those skilled in the art should take the specification as a whole. The technical solutions in the embodiments can also be appropriately combined to form other implementations which are comprehensible for those skilled in the art.

What is claimed is:

1. A computational method for considering contribution of biological activity to a cochlear sensory amplification mechanism, comprising:
establishing an analytical model, which considers periodic variation of a stiffness of a cochlear basement membrane in space and time, and characterizes a relationship between physical parameters of a human cochlear and an amplitude of the basement membrane;
performing stability analysis of the analytical model, using non-periodic solution and periodic solution to obtain resonant characteristics of the analytical model;
obtaining a stiffness of a basement membrane of a non-hearing-impaired person by using the analytical model with a vertical displacement of the basement membrane of the non-hearing-impaired person as an input of the analytical model;
obtaining a stiffness of a basement membrane of a hearing-impaired person by using the analytical model with a vertical displacement of the basement membrane of the hearing-impaired person as an input of the analytical model; and
determining a hardening degree of the basement membrane of the hearing-impaired person according to the stiffness of the basement membrane of the non-hearing-impaired person and the stiffness of the basement membrane of the hearing-impaired person.

2. The computational method for considering contribution of biological activity to a cochlear sensory amplification mechanism according to claim 1, wherein a process of establishing the analytical model comprises calculating a volume force f:
$$f ( x , t ) = {\int }_{0}^{L} K ( s , t ) ( {X}_{0} - X ) δ ( x - X ) ds ,$$
in the formula, δ(x) represents a two-dimensional Dirac Delta function, X(s,t) represents position parameters of the basement membrane (BM) in a Lagrangian coordinate, and X₀(s)=(s, 0) represents an equilibrium position;
a stiffness parameter of the BM is assumed to vary with time and space, and is expressed by a function as:
$$K ( s , t ) = σ {e}^{- λ s} ( 1 + 2 τ sin ( ω t ) ) ,$$
in the formula, σ is an elastic stiffness constant in a mean time interval, λ describes a variation of stiffness along a BM space, and from previous experimental data, the stiffness of the BM is found to present an exponential variation rule along a length; an exponential function is used to describe the spatial variation of the stiffness; a periodic variation rule of the stiffness in a periodic vibration process of the BM is described by an amplitude parameter τ and a frequency ω;
according to coupled vibration interface conditions between fluid and the BM, the following is derived:
$$\frac{\partial X}{\partial t} = u ( X , t ) = {\int }_{Ω} u ( x , t ) δ ( x - X ) dx ,$$
dimensionless processing is used, and a dimensionless quantity is as follows:
$$x = \frac{L \tilde{x}}{π} , t = \frac{\tilde{t}}{ω} , u = {U}_{c} \tilde{u} , p = {P}_{c} \tilde{p} , X = \frac{L \tilde{X}}{π} , s = \frac{L \tilde{s}}{π} ,$$
in the formula, a subject on a wavy line is the dimensionless quantity, Uc and Pc respectively represent characteristic scales of velocity and pressure, and by substituting the dimensionless quantity, the following is obtained:
$$\frac{\partial \tilde{u}}{\partial \tilde{t}} + \tilde{u} \cdot \tilde{∇} \tilde{u} = - \tilde{∇} \tilde{p} + v \tilde{Δ} \tilde{u} + \tilde{f}$$
$$\tilde{∇} \cdot \tilde{u} = 0$$
$$\tilde{f} ( \tilde{x} , \tilde{t} ) = {\int }_{0}^{π} \tilde{K} ( {\tilde{X}}_{0} - \tilde{X} ) \tilde{δ} ( \tilde{x} - \tilde{X} ) d \tilde{s} ,$$
$$\tilde{K} ( \tilde{s} , \tilde{t} ) = κ {e}^{- α \tilde{s}} ( 1 + 2 τ sin \tilde{t} )$$
$$\frac{\partial \tilde{X}}{\partial \tilde{t}} = u ( \tilde{X} , \tilde{t} )$$
the characteristic scales of velocity and pressure are expressed as:
$${U}_{c} = \frac{L ω}{π} , {P}_{c} = \frac{ρ {ω}^{2} {L}^{2}}{{π}^{2}} ,$$
parameters in equations are expressed as:
$$v = \frac{μ {π}^{2}}{ρ {L}^{2} ω} , κ = \frac{σ π}{ρ L {ω}^{2}} , α = \frac{λ L}{π} ,$$
a system equation of the analytical model is:
$$\frac{\partial u}{\partial t} = - ∇ p + v Δ u ,$$
$$∇ \cdot u = 0$$
and there are the following conditions:
$$p = - κ {e}^{- α x} ( 1 + 2 τ sin t ) h ( x , t )$$
$$u ( x , 0 , t ) = 0 ,$$
$$v ( x , 0 , t ) = \frac{\partial h}{\partial t}$$
h(x,t) represents vertical displacement of the BM, p(x,t)=p(x,0⁺,t)−p(x,0⁻¹,t) represents a pressure difference on the BM, and u(x,y,t) and v(x,y,t) represent vertical and vertical velocities respectively.

3. The computational method for considering contribution of biological activity to a cochlear sensory amplification mechanism according to claim 1, wherein the solution of the analytical model satisfies the following forms:
$$u ( x , t ) = {e}^{γ t} P ( x , t ) ,$$
in the formula, the function P(x,t) represents a periodic function with a period of 2π, an exponential factor determines the stability of the solution when t−>∞, and series representation of P(x,t) is performed in an interval of [−π,π] to obtain:
$$u ( x , y , t ) = {e}^{γ t} {\sum }_{n = - ∞}^{∞} {\sum }_{k = - ∞}^{∞} {u}_{k}^{n} ( y ) {e}^{int} {e}^{ikx}$$
$$v ( x , y , t ) = {e}^{γ t} {\sum }_{n = - ∞}^{∞} {\sum }_{k = - ∞}^{∞} {v}_{k}^{n} ( y ) {e}^{int} {e}^{ikx} ,$$
$$p ( x , y , t ) = {e}^{γ t} {\sum }_{n = - ∞}^{∞} {\sum }_{k = - ∞}^{∞} {p}_{k}^{n} ( y ) {e}^{int} {e}^{ikx}$$
$$h ( x , y , t ) = {e}^{γ t} {\sum }_{n = - ∞}^{∞} {\sum }_{k = - ∞}^{∞} {h}_{k}^{n} ( y ) {e}^{int} {e}^{ikx}$$
in the formula, Fourier series expansion in space and time is performed on P(x,t); parameters to be solved in the above equation are Fourier coefficients uₖⁿ, vₖⁿ, and pₖⁿ varying along a Y-axis, and substitution is performed to obtain an equation of pressure expressed as:
$$Δ p = {\sum }_{n , k = - ∞}^{∞} ( - {k}^{2} {p}_{k}^{n} ( y ) + {p}_{k}^{{n}^{′}} ( y ) ) {ε}_{k}^{n} = 0 ,$$
in the formula, εₖⁿ(x,t)=e^{[(γ+in)t+ikx]}, due to linear independence of εₖⁿ(x,t), there is: −k²pₖⁿ(y)+pₖ^{n″}(y)=0,
by solving the above equation, the following is obtained:
$${p}_{k}^{n} ( y ) = \{ \begin{matrix}{α}_{k}^{n} {e}^{ky} , & y < 0 \\ {b}_{k}^{n} {e}^{- k y} , & y > 0\end{matrix} , and$$
$${\sum }_{n , k = - ∞}^{∞} ( γ + in ) {v}_{k}^{n} ( y ) {ε}_{k}^{n} = {\sum }_{n , k = - ∞}^{∞} ( - {p}_{k}^{{n}^{′}} ( y ) - {vk}^{2} {v}_{k}^{n} ( y ) + {vv}_{k}^{{n}^{″}} ( y ) ) {ε}_{k}^{n} ,$$
after further derivation, an ordinary differential equation is obtained as follows:
$${v}_{k}^{{n}^{″}} ( y ) - {( {β}_{k}^{n} )}^{2} {v}_{k}^{n} ( y ) = \frac{1}{v} {p}_{k}^{{n}^{′}} ( y ) ,$$
in the formula,
$${β}_{k}^{n} = \sqrt{\frac{γ + in}{v} + {k}^{2}} ,$$
supposing γ+in≠0 and k≠0, then a solution of the above formula is:
$${v}_{k}^{n} ( y ) = \frac{1}{2 v {β}_{k}^{n}} \{ \begin{matrix}- \frac{2 k {β}_{k}^{n}}{{( {β}_{k}^{n} )}^{2} - {k}^{2}} {α}_{k}^{n} {e}^{ky} + ( \frac{k}{{β}_{k}^{n} - k} {α}_{k}^{y} + \frac{k}{{β}_{k}^{n} + k} {b}_{k}^{y} ) {e}^{{β}_{k}^{n} y} , & y < 0 \\ \frac{2 k {β}_{k}^{n}}{{( {β}_{k}^{n} )}^{2} - {k}^{2}} {b}_{k}^{n} {e}^{- ky} + ( \frac{k}{{β}_{k}^{n} - k} {b}_{k}^{y} + \frac{k}{{β}_{k}^{n} + k} {α}_{k}^{y} ) {e}^{- {β}_{k}^{n} y} , & y > 0\end{matrix} ,$$
according to continuity, an equation is obtained:
ikuₖⁿ(y)+νₖ^{n′}(y)=0,
then the equation is solved to obtain:
$${u}_{k}^{n} ( y ) = \frac{- i}{2 kv} \{ \begin{matrix}- \frac{2 {k}^{2}}{{( {β}_{k}^{n} )}^{2} - {k}^{2}} {α}_{k}^{n} {e}^{ky} + ( \frac{k}{{β}_{k}^{n} - k} {α}_{k}^{y} + \frac{k}{{β}_{k}^{n} + k} {b}_{k}^{y} ) {e}^{{β}_{k}^{n} y} , & y < 0 \\ - \frac{2 k {β}_{k}^{n}}{{( {β}_{k}^{n} )}^{2} - {k}^{2}} {b}_{k}^{n} {e}^{- ky} + ( \frac{k}{{β}_{k}^{n} - k} {b}_{k}^{y} + \frac{k}{{β}_{k}^{n} + k} {α}_{k}^{y} ) {e}^{- {β}_{k}^{n} y} , & y > 0\end{matrix} ,$$
according to the continuity, interface boundary conditions are derived:
$${u}_{k}^{n} ( {0}^{+} ) = {u}_{k}^{n} ( {0}^{-} ) = \frac{1}{2 v} ( \frac{1}{{β}_{k}^{n} + k} {α}_{k}^{n} + \frac{1}{{β}_{k}^{n} + k} {b}_{k}^{n} ) = 0 ,$$
$${v}_{k}^{n} ( {0}^{+} ) = {v}_{k}^{n} ( {0}^{-} ) = \frac{1}{2 v {β}_{k}^{n}} ( \frac{- k}{{β}_{k}^{n} + k} {α}_{k}^{n} + \frac{k}{{β}_{k}^{n} + k} {b}_{k}^{n} ) = ( γ + in ) {h}_{k}^{y}$$
after further derivation, the following is obtained:
$${α}_{k}^{n} = - v ( γ + in ) \frac{{β}_{k}^{n} + k}{k} {β}_{k}^{n} {h}_{k}^{n} ,$$
$${b}_{k}^{n} = v ( γ + in ) \frac{{β}_{k}^{n} + k}{k} {β}_{k}^{n} {h}_{k}^{n}$$
substitution is performed to obtain:
$${\sum }_{n , k = - ∞}^{∞} 2 v ( γ + in ) \frac{{β}_{k}^{n} + k}{k} {β}_{k}^{n} {h}_{k}^{n} {ε}_{k}^{n} = {\sum }_{n , k = - ∞}^{∞} - κ {e}^{- α x} ( 1 + 2 τ sin t ) {h}_{k}^{n} {ε}_{k}^{n} ,$$
when γ+in=0 and k=0, the above equation is simplified as:
$$0 = {\sum }_{n , k = - ∞}^{∞} - κ {e}^{- α x} ( 1 + 2 τ sin t ) {h}_{k}^{n} {ε}_{k}^{n} ,$$
after Fourier expansion of the exponential function and sine function in the above formula, there is 1+2τ sin t=1−iτe^{it}+iτe^{-it};
even function periodic expansion of the exponential function is performed, and when γ+in≠0 and k≠0, there is:
$${\sum }_{n , k = - ∞}^{∞} 2 v ( γ + in ) \frac{{β}_{k}^{n} + k}{k} {β}_{k}^{n} {h}_{k}^{n} {ε}_{k}^{n} = {\sum }_{n , k = - ∞}^{∞} - κ ( {\sum }_{j = - ∞}^{∞} {c}_{j} {e}^{ijx} ) ( 1 - i τ {e}^{it} + i τ {e}^{- it} ) {h}_{k}^{n} {ε}_{k}^{n} ,$$
when γ+in=0 and k=0, there is:
$$0 = {\sum }_{n , k = - ∞}^{∞} - κ ( {\sum }_{j = - ∞}^{∞} {c}_{j} {e}^{ijx} ) ( 1 - i τ {e}^{it} + i τ {e}^{- it} ) {h}_{k}^{n} {ε}_{k}^{n} ,$$
wherein
$${c}_{j} = α \frac{1 - {( - 1 )}^{j} {e}^{- α π}}{π ( {α}^{2} + {j}^{2} )}$$
is a Fourier coefficient of the exponential function; and by sorting out coefficients of a term εₖⁿ, the following is obtained:
$$\frac{2 {v}^{2}}{κ} ( {β}_{k}^{n} - k ) {( {β}_{k}^{n} + k )}^{2} \frac{{β}_{k}^{n}}{k} {h}_{k}^{n} + {\sum }_{j = - ∞}^{∞} {c}_{k - j} {h}_{j}^{n} = i τ {\sum }_{j = - ∞}^{∞} {c}_{k - j} ( {h}_{j}^{n - 1} - {h}_{j}^{n + 1} ) ,$$
when γ+in=0 and k=0, there is:
$${\sum }_{j = - ∞}^{∞} {c}_{k - j} {h}_{j}^{n} = i τ {\sum }_{j = - ∞}^{∞} {c}_{k - j} ( {h}_{j}^{n - 1} - {h}_{j}^{n + 1} ) ,$$
in order to ensure space symmetry of even functions of solutions, there are:
$${h}_{- k}^{n} = {h}_{k}^{n}$$
$${h}_{k}^{- n} = \{ \begin{matrix}{\overline{h}}_{k}^{n} , & γ = 0 \\ {\overline{h}}_{k}^{n - 1} , & γ = \frac{1}{2} i\end{matrix} ,$$
for the solution u(x,t)=e^{γt}P(x,t) the following periodic conditions are implied:
u(x,t+2πn)=e^{γ(t+2πn)}P(x,t)=ξⁿu(x,t),
if γ=0, then ξ=1, and there is:
u(x,t+2π)=u(x,t)
the above formula is a harmonic solution with a period of 2π; if γ=½*i, then ξ=−1, and there is:
u(x,t+2π)=−u(x,t),u(x,t+4π)=u(x,t)
the system equation of the analytical model is reduced, n=0, 1, . . . , N, and k=1, 2, . . . , M, and by matrix representation, the following is obtained:
$$A \vec{h} = τ B \vec{h} , wherein$$
$$\vec{h} = {[ … , Re ( {h}_{k}^{n} ) , Im ( {h}_{k}^{n} ) , Re ( {h}_{k + 1}^{n} ) , Im ( {h}_{k + 1}^{n} ) , … ]}^{T} ,$$
the above equations comprise 2*M*(N+1) unknown coefficients to be solved, and A and B are skew diagonal matrices; and the skew diagonal matrix A is expressed as A=diag(A⁰, A¹, . . . , A^{N}) and has the following forms:
$${A}^{n} = [ \begin{matrix}{C}_{1 , 1} + {D}_{1}^{n} & {C}_{1 , 2} & … & {C}_{1 , M} \\ {C}_{2 , 1} & {C}_{2 , 2} + {D}_{2}^{n} & … & {C}_{2 , M} \\ ⋮ & ⋮ & ⋱ & ⋮ \\ {C}_{M , 1} & {C}_{M , 2} & … & {C}_{M , M} + {D}_{M}^{n}\end{matrix} ] ,$$
wherein
$${C}_{k , j} = [ \begin{matrix}{C}_{k - j} + {c}_{k + j} & 0 \\ 0 & {c}_{k + j} + {c}_{k + j}\end{matrix} ] , and$$
$${D}_{k}^{n} = \frac{2 {v}^{2}}{κ k} [ \begin{matrix}Re \{ ( {β}_{k}^{n} - k ) {( {β}_{k}^{n} + k )}^{2} {β}_{k}^{n} \} & - Im \{ ( {β}_{k}^{n} - k ) {( {β}_{k}^{n} + k )}^{2} {β}_{k}^{n} \} \\ Im \{ ( {β}_{k}^{n} - k ) {( {β}_{k}^{n} + k )}^{2} {β}_{k}^{n} \} & Re \{ ( {β}_{k}^{n} - k ) {( {β}_{k}^{n} + k )}^{2} {β}_{k}^{n} \}\end{matrix} ] ,$$
the triangular skew diagonal matrix B has the following forms:
$$B = [ \begin{matrix}\hat{B} & \hat{B} & & & \\ \hat{B} & 0 & - \hat{B} & & \\ & ⋱ & ⋱ & ⋱ & \\ & & \hat{B} & 0 & - \hat{B} \\ & & & \hat{B} & 0\end{matrix} ] , wherein$$
$$\hat{B} = [ \begin{matrix}{\hat{C}}_{1 , 1} & {\hat{C}}_{1 , 2} & … & {\hat{C}}_{1 , M} \\ {\hat{C}}_{2 , 1} & {\hat{C}}_{2 , 2} & … & {\hat{C}}_{2 , M} \\ ⋮ & ⋮ & ⋱ & ⋮ \\ {\hat{C}}_{M , 1} & \hat{C} {C}_{M , 2} & … & {\hat{C}}_{M , M}\end{matrix} ] ,$$
$${\hat{C}}_{k , j} = [ \begin{matrix}0 & - {c}_{k - j} + {c}_{k + j} \\ {c}_{k - j} + {c}_{k + j} & 0\end{matrix} ] ,$$
and
both A and B matrices are known, that is,
$${A}^{- 1} B \vec{h} = \frac{1}{τ} \vec{h} ,$$
an eigenvalue of the stability solution in the above equation is 1/τ.

4. The computational method for considering contribution of biological activity to a cochlear sensory amplification mechanism according to claim 1, wherein non-periodic solution comprises:
when τ=0 in the stiffness function of the BM, the stiffness function is a non-periodic function, the solution of the equation is stable, and the Fourier coefficient of the solution satisfies:
$$\frac{2 ϕ γ}{k v} \sqrt{\frac{γ}{v} + {k}^{2}} ( k + \sqrt{\frac{γ}{v} + {k}^{2}} ) {h}_{k}^{0} + {\sum }_{j = - ∞}^{∞} {c}_{k - j} {h}_{j}^{0} = 0 ,$$
when k=0, there is:
$${\sum }_{j = - ∞}^{∞} {c}_{k - j} {h}_{j}^{0} = 0 ,$$
in the above formula, ϕ=ν²/κ=π³μ²/(ρσL³) represents a ratio of a fluid viscosity resistance to an elastic force of the BM; and the above equation is expressed as
$$T {\vec{h}}^{0} = 0 ,$$
and the T matrix depends on parameters ϕ, γ, and α, and a condition for existence of nonsingular solutions in the analytical model is to satisfy det (T)=0, and when ϕ and α are given, γ is obtained by solution.

5. The computational method for considering contribution of biological activity to a cochlear sensory amplification mechanism according to claim 1, wherein periodic solution comprises solving formulas
$${\sum }_{j = - ∞}^{∞} {c}_{k - j} {h}_{j}^{0} = 0 ,$$
to obtain τ and corresponding eigenvectors; and on this basis, the periodic solution h(x,t) is solved with a formula
$$\frac{2 {v}^{2}}{κ} ( {β}_{k}^{n} - k ) {( {β}_{k}^{n} + k )}^{2} \frac{{β}_{k}^{n}}{k} {h}_{k}^{n} + {\sum }_{j = - ∞}^{∞} {c}_{k - j} {h}_{j}^{n} = i τ {\sum }_{j = - ∞}^{∞} {c}_{k - j} ( {h}_{j}^{n - 1} - {h}_{j}^{n + 1} ) and$$
$${\sum }_{j = - ∞}^{∞} {c}_{k - j} {h}_{j}^{n} = i τ {\sum }_{j = - ∞}^{∞} {c}_{k - j} ( {h}_{j}^{n - 1} - {h}_{j}^{n + 1} )$$
